(19) United States
(12) Patent Application Publication
(10) Pub. No.: US 2008/0118562 A1
(43) Pub. Date: May 22, 2008

(54) Bis(thiohydrazide amides) formulation

(76) Inventor: Keizo Koya, Chestnut Hill, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/900,201

(22) Filed: Sep. 10, 2007

Related U.S. Application Data
(60) Provisional application No. 60/843,941, filed on Sep. 11, 2006.

(51) Int. Cl. A61K 47/48; A61P 35/00; A61K 31/16

(52) U.S. Cl. 424/486; 514/599; 424/484

(57) ABSTRACT

Disclosed herein are compositions and methods useful for the in vivo delivery bis(thiohydrazide amides), encased in a polymeric biocompatible shell.

### RELATED APPLICATION(S)

This application claims the benefit of U.S. Provisional Application No. 60/843,941 filed on Sep. 11, 2006. The entire teachings of the above application are incorporated herein by reference.

### BACKGROUND OF THE INVENTION

The major limitation of certain anti-cancer drugs is their poor solubility in biocompatible solvents. Consequently, typical formulations of anti-cancer drugs contain ingredients which cause severe side effects in patients and often require premedication to reduce the hypersensitivity associated with these formulations. Further, to reduce side effects these formulations are typically diluted, resulting in large volumes of infusion to the patient of up to 1 liter and infusion times ranging from 3 hours to 24 hours.

Thus, there is a need for an alternative less toxic formulations for anti-cancer drugs.

### SUMMARY OF THE INVENTION

The present invention relates to composition, comprising biocompatible, water-soluble polymeric particles for delivery of bis(thiohydrazide amides).

The bis(thio-hydrazide amide) used in the compositions and methods of the present invention are represented by Structural Formula I:

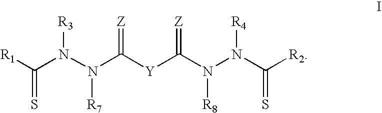

Y is a covalent bond or an optionally substituted straight chained hydrocarbyl group, or, Y, taken together with both >C=Z groups to which it is bonded, is an optionally substituted aromatic group.

R₁-R₄ are independently —H, an optionally substituted aliphatic group, an optionally substituted aryl group, or R₁ and R₃ taken together with the carbon and nitrogen atoms to which they are bonded, and/or R₂ and R₄ taken together with the carbon and nitrogen atoms to which they are bonded, form a non-aromatic ring optionally fused to an aromatic ring.

R₇-R₈ are independently —H, an optionally substituted aliphatic group, or an optionally substituted aryl group.

Z is O or S.

In certain embodiments the bis(thio-hydrazide amide) used in the compositions and methods are substantially or completely encased in a polymeric shell. In one embodiment the present invention is a composition comprising a compound represented by the following Structural Formula:

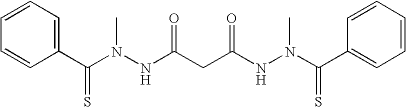

or a pharmaceutically acceptable salt thereof, wherein the compound is substantially or completely encased in a biocompatible polymeric shell, wherein the biocompatible polymeric shell is albumin substantially crosslinked by disulfide bonds.

In one embodiment the present invention is a composition prepared by subjecting an organic phase comprising a bis(thiohydrazide amide), and an aqueous medium comprising a biocompatible polymer, to sonication conditions for a time sufficient to promote crosslinking of said biocompatible polymer by disulfide bonds to produce a polymeric shell encasing substantially or completely the bis(thiohydrazide amide).

In one embodiment the present invention is a composition prepared by subjecting an organic phase comprising a bis(thiohydrazide amide), and an aqueous medium comprising a biocompatible polymer, to high shear conditions in a high pressure homogenizer at a pressure in the range of about 100 up to about 100,000 psi for a time sufficient to promote crosslinking of said biocompatible polymer by disulfide bonds to produce a polymeric shell encasing substantially or completely the compound.

In one embodiment the present invention is a method of making a composition comprising a bis(thiohydrazide amide) substantially or completely encased within a polymeric shell, comprising subjecting an organic phase comprising the bis(thiohydrazide amide), and an aqueous medium comprising a biocompatible polymer, to sonication conditions for a time sufficient to promote crosslinking of said biocompatible polymer by disulfide bonds to produce the polymeric shell encasing substantially or completely the bis(thiohydrazide amide).

In one embodiment the present invention is a method of making a composition comprising a bis(thiohydrazide amide) substantially or completely encased within a polymeric shell, comprising subjecting an organic phase comprising the bis(thiohydrazide amide), and an aqueous medium comprising a biocompatible polymer, to high shear conditions in a high pressure homogenizer at a pressure in the range of about 100 up to about 100,000 psi for a time sufficient to promote crosslinking of said biocompatible polymer by disulfide bonds to produce a polymeric shell encasing substantially or completely the compound.

In one embodiment the present invention is a method of treating a subject with cancer comprising administering to the subject an effective amount of a bis(thiohydrazide amide) substantially or completely encased within a polymeric shell.

In one embodiment the present invention is a method of treating a subject with cancer comprising administering to the subject an effective amount of a bis(thiohydrazide amide) and an effective amount of an anti-cancer agent wherein the bis(thiohydrazide amide) is substantially or completely encased within a polymeric shell.

In one embodiment the present invention is a method of treating a subject with cancer comprising administering to the subject an effective amount of a bis(thiohydrazide amide) and an effective amount of an anti-cancer agent wherein the bis(thiohydrazide amide) and the anti-cancer agent are substantially or completely encased within a polymeric shell.

The disclosed compositions, in general will be less toxic than currently available formulations and will not require premedication of patients. The polymeric shell containing bis(thiohydrazide amides) in general allows for the delivery of high doses of the bis(thiohydrazide amides) in relatively small volumes. This would minimize patient discomfort at receiving large volumes of fluid and minimizes hospital stays. In addition, the walls of the polymeric shell are generally completely degradable in vivo by proteolytic enzymes (e.g., when the polymer is a protein), resulting in no side effects from the delivery system as is the case with current formulations.

### DETAILED DESCRIPTION OF THE INVENTION

The present invention relates to composition, comprising biocompatible, water-soluble polymeric particles for delivery of bis(thiohydrazide amides) to a subject. In one embodiment, the compositions are in the form of particles comprising bis(thiohydrazide amides) encased in a polymeric shell. In general, the polymeric shell is formulated from a biocompatible polymer.

In one embodiment of the present invention bis(thiohydrazide amides) can be delivered in the form of microparticles or nanoparticles that are suitable for parenteral administration in aqueous suspension.

In one embodiment, particles of bis(thiohydrazide amides) are contained within a shell having a cross-sectional diameter of less than about 100 micron, less than about 50, less than about 20 microns, less than about 10 microns, less than about 5 microns, less than about 1 microns. A cross-sectional diameter of less than 5 microns is more preferred, while a cross-sectional diameter of less than 1 micron is presently the most preferred for the intravenous route of administration.

In one embodiment the polymeric shell produced by the invention process is relatively thin compared to the diameter of the particle. It one embodiment the “shell thickness” of the polymeric coat is less than about 500 nm, less than about 100 nm, less than about 50 nm, less than about 25 nm, or approximately 25 nanometers for a coated particle having a diameter of 1 micron (1000 nanometers).

A number of biocompatible materials may be employed in the practice of the present invention for the formation of a polymeric shell. As used herein, the term “biocompatible” describes a substance that does not appreciably alter or affect in any adverse way, the biological system into which it is introduced.

Essentially any polymer, natural or synthetic, bearing sulfhydryl groups or disulfide bonds within its structure may be utilized for the preparation of a disulfide crosslinked shell. The sulfhydryl groups or disulfide linkages may be preexisting within the polymer structure or they may be introduced by a suitable chemical modification. For example, naturally occurring biocompatible materials such as proteins, polypeptides, oligopeptides, polynucleotides, polysaccharides (e.g., starch, cellulose, dextrans, alginates, chitosan, pectin, hyaluronic acid, and the like), lipids, and so on, are candidates for such modification.

As examples of suitable biocompatible polymers, naturally occurring or synthetic proteins may be employed, so long as such proteins have sufficient cysteine residues within their amino acid sequences so that crosslinking (through disulfide bond formation, for example, as a result of oxidation during sonication) can occur. Examples of suitable proteins include albumin (which contains 35 cysteine residues), insulin (which contains 6 cysteines), hemoglobin (which contains 6 cysteine residues per α₂β₂ unit), lysozyme (which contains 8 cysteine residues), immunoglobulins, α-2-macroglobulin, fibronectin, vitronectin, fibrinogen, casein and the like, as well as combinations of any two or more thereof.

A presently preferred protein for use in the formation of a polymeric shell is albumin. Optionally, proteins such as α-2-macroglobulin, known opsonin, could be used to enhance uptake of the shell encased particles by macrophage-like cells, or to enhance the uptake of the shell encased particles into the liver and spleen. Other ligands such as glycoproteins may also enhance uptake into certain tissues. Other functional proteins, such as antibodies or enzymes, which could facilitate targeting of bis(thiohydrazide amide) to a desired site, can also be used in the formation of the polymeric shell.

In one embodiment, the polymer is human serum albumin (HSA).

Similarly, synthetic polymers are also good candidates for preparation of the particles of the present invention. Examples include polyalkylene glycols (e.g., linear or branched chain), polyvinyl alcohol, polyacrylates, polyhydroxyethyl methacrylate, polyacrylic acid, polyethyloxazoline, polyacrylamides, polyisopropyl acrylamides, polyvinyl pyrrolidinone, polylactide/glycolide and the like, and combinations thereof, are good candidates for the biocompatible polymer in the invention formulation.

Exemplary unmodified synthetic polypeptides contemplated for use in the practice of the present invention are such materials as synthetic polyamino acids (optionally containing cysteine residues and/or disulfide groups), polyvinyl alcohol, polyhydroxyethyl methacrylate, polyacrylic acid, polyethyloxazoline, polyacrylamide, polyvinyl pyrrolidinone, polyalkylene glycols, polylactides, polyglycolides, polycaprolactones, or copolymers thereof, and the like, and suitable combinations of any two or more thereof.

In addition, the unmodified synthetic polypeptides contemplated for use in the practice of the present invention listed above are good candidates for chemical modification (for example, by the introduction of sulfhydryl and/or disulfide linkages) and coating formation (e.g., shell formation, caused, for example, by the crosslinking thereof). Thus, for example, contemplated for use in the practice of the present invention are such materials as polyvinyl alcohol modified to contain free sulfhydryl groups and/or disulfide groups; polyhydroxyethyl methacrylate modified to contain free sulfhydryl groups and/or disulfide groups; polyacrylic acid modified to contain free sulfhydryl groups and/or disulfide groups; polyethyloxazoline modified to contain free sulfhydryl groups and/or disulfide groups; polyacrylamide modified to contain free sulfhydryl groups and/or disulfide groups; polyvinyl pyrrolidinone modified to contain free sulfhydryl groups and/or disulfide groups; polyalkylene glycols modified to contain free sulfhydryl groups and/or disulfide groups; polylactides, polyglycolides, polycaprolactones, or copolymers thereof, modified to contain free sulfhydryl groups and/or disulfide groups; as well as mixtures of any two or more thereof.

Suitable mixtures of any two or more of the foregoing biocompatible polymers are also contemplated for use in the practice of the present invention.

Biocompatible polymer(s) (i.e., the stabilizing agent) is typically added at a concentration in the range of about 0.001 to about 50% (w/v), more preferably in the range of about 0.1% to about 25% (w/v), with a presently preferred range of about 0.5% to about 5% (w/v), as measured in the final mixture prior to evaporation and lyophilization.

These biocompatible materials may also be employed in several physical forms such as gels, crosslinked or uncrosslinked to provide matrices from which the bis(thiohydrazide amides) may be released by diffusion and/or degradation of the matrix. Temperature sensitive materials may also be utilized as the dispersing matrix for the invention formulation. Thus for example, the bis(thiohydrazide amide) particles may be injected in a liquid formulation of the temperature sensitive material (e.g., copolymers of polyacrylamides or copolymers of polyalkylene glycols and polylactide/glycolides) which gel at the tumor site and provide slow release of bis(thiohydrazide amides). The bis(thiohydrazide amides) formulation may be dispersed into a matrix of the above mentioned biocompatible polymers to provide a controlled release formulation of bis(thiohydrazide amide), which through the properties of the particles (albumin associated with bis(thiohydrazide amides)) in general may result in lower toxicity.

In addition, the polymeric shell can optionally be modified by a suitable agent, wherein the agent is associated with the polymeric shell through an optional covalent bond. Covalent bonds contemplated for such linkages include ester, ether, urethane, diester, amide, secondary or tertiary amine, phosphate ester, sulfate ester, and the like bonds. Suitable agents contemplated for this optional modification of the polymeric shell include synthetic polymers (polyalkylene glycols (e.g., linear or branched chain polyethylene glycol), polyvinyl alcohol, polyhydroxyethyl methacrylate, polyacrylic acid, polyethyloxazoline, polyacrylamide, polyvinyl pyrrolidinone, and the like), phospholipids (such as phosphatidyl choline (PC), phosphatidyl ethanolamine (PE), phosphatidyl inositol (PI), sphingomyelin, and the like), proteins (such as enzymes, antibodies, and the like), polysaccharides (such as starch, cellulose, dextrans, alginates, chitosan, pectin, hyaluronic acid, and the like), chemical modifying agents (such as pyridoxal 5′-phosphate, derivatives of pyridoxal, dialdehydes, diaspirin esters, and the like), or combinations thereof.

Variations in the polymeric shell are possible. For example, a small amount of PEG containing sulfhydryl groups could be included with the polymer. Upon exposure to ultrasonic irradiation as described herein, the PEG is crosslinked into the polymer and forms a component of the polymeric shell. Alternatively, PEG can be linked to the polymeric shell following the preparation of the shell (rather than being included as part of the media from which the shell is prepared).

Useful for the modification of the polymeric shell are electrophilic PEG derivatives including PEG-imidazoles, succinimidyl succinates, nitrophenyl carbonates, tresylates, and the like; nucleophilic PEG derivatives including PEG-amines, amino acid esters, hydrazides, thiols, and the like. The PEG-modified polymeric shell will be expected to persist in the circulation for longer periods than their unmodified counterparts. The modification of polymeric shell with PEG may be performed before formation of the shell, or following formation thereof. The currently preferred technique is to modify the polymeric shell after formation thereof. Other polymers including dextran, alginates, hydroxyethyl starch, and the like, may be utilized in the modification of the polymeric shell.

PEG is known for its nonadhesive character and has been attached to proteins and enzymes to increase their circulation time in vivo [Abuchowski et al., J. Biol. Chem. Vol. 252:3578 (1977)]. PEG has also been attached to phospholipids forming the lipidic bilayer in liposomes to reduce their uptake and prolong lifetimes in vivo [Klibanov et al., FEBS Letters Vol. 268:235 (1990)]. Thus the incorporation of PEG into the walls of crosslinked protein shells alters their blood circulation time. This property can be exploited to maintain higher blood levels of bis(thiohydrazide amides) and prolonged release times for the bis(thiohydrazide amides).

In the preparation of invention compositions, one can optionally employ a dispersing agent to suspend or dissolve the bis(thiohydrazide amides) within the polymer shell. Dispersing agents contemplated for use in the practice of the present invention include any nonaqueous liquid that is capable of suspending or dissolving the bis(thiohydrazide amides), but does not chemically react with either the polymer employed to produce the shell, or the bis(thiohydrazide amide) itself. Examples include water, vegetable oils (e.g., soybean oil, mineral oil, corn oil, rapeseed oil, coconut oil, olive oil, safflower oil, cotton seed oil, and the like), aliphatic, cycloaliphatic, or aromatic hydrocarbons having 4-30 carbon atoms (e.g., n-dodecane, n-decane, n-hexane, cyclohexane, toluene, benzene, and the like), aliphatic or aromatic alcohols having 1-30 carbon atoms (e.g., octanol, and the like), aliphatic or aromatic esters having 2-30 carbon atoms (e.g., ethyl caprylate (octanoate), and the like), alkyl, aryl, or cyclic ethers having 2-30 carbon atoms (e.g., diethyl ether, tetrahydrofuran, and the like), alkyl or aryl halides having 1-30 carbon atoms (and optionally more than one halogen substituent, e.g., CH₃Cl, CH₂Cl₂, CHCl₃, CH₂ClCH₂Cl, and the like), ketones having 3-30 carbon atoms (e.g., acetone, methyl ethyl ketone, and the like), polyalkylene glycols (e.g., polyethylene glycol, and the like), or combinations thereof.

Especially preferred combinations of dispersing agents include volatile liquids such as dichloromethane, chloroform, ethyl acetate, benzene, and the like (i.e., solvents that have a high degree of solubility for the bis(thiohydrazide amide), and are soluble in the other dispersing agent employed), along with a less volatile dispersing agent. When added to the other dispersing agent, these volatile additives help to drive the solubility of the bis(thiohydrazide amide) into the dispersing agent. This is desirable sine this step is usually time consuming. Following dissolution, the volatile component may be removed by evaporation (optionally under vacuum).

Variations on the general theme of bis(thiohydrazide amides) encased within the polymeric shell are possible. A suspension of fine particles of bis(thiohydrazide amides) in a biocompatible dispersing agent could be used (in place of a biocompatible dispersing agent containing dissolved or suspended bis(thiohydrazide amides)) to produce a polymeric shell containing dispersing agent-suspended particles of bis(thiohydrazide amides). In other words, the polymeric shell could contain a saturated solution of bis(thiohydrazide amides) in dispersing agent. Another variation is a polymeric shell containing a solid core of bis(thiohydrazide amides) produced by initially dissolving the bis(thiohydrazide amides) in a volatile organic solvent (e.g. benzene), forming the polymeric shell and evaporating the volatile solvent under vacuum, e.g., in an evaporator, spray drier or freeze-drying the entire suspension. This results in a structure having a solid core of bis(thiohydrazide amides) surrounded by a polymer coat. This latter method is particularly advantageous for delivering high doses of bis(thiohydrazide amides) in a relatively small volume. In some cases, the biocompatible material forming the shell about the core could itself be a therapeutic or diagnostic agent. In other cases, the polymer forming the shell could participate in the delivery of the bis(thiohydrazide amides).

Particles of bis(thiohydrazide amides) substantially completely contained within a polymeric shell, or associated therewith, prepared as described herein, are delivered neat, or optionally dissolved, dispersed or as a suspension in a biocompatible medium. This medium may be selected from water, buffered aqueous media, saline, buffered saline, optionally buffered solutions of amino acids, optionally buffered solutions of proteins, optionally buffered solutions of sugars, optionally buffered solutions of carbohydrates, optionally buffered solutions of vitamins, optionally buffered solutions of synthetic polymers, lipid-containing emulsions, and the like.

In one embodiment, since polymers such as, for example, HSA are freely soluble in water, bis(thiohydrazide amides) particles as described herein can be reconstituted to any desired concentration of limited only by the solubility limits for HSA.

In accordance with the present invention, there is provided submicron particles in powder form, which can easily be reconstituted in water or saline. The powder is obtained after removal of water by lyophilization. Human serum albumin serves as the structural component of the particles of the present invention, and also as a cryoprotectant and reconstitution aid. The preparation of particles filterable through a 0.22 micron filter according to the invention method as described herein, followed by drying or lyophilization, produces a sterile solid formulation useful for intravenous injection.

While it is recognized that particles produced according to the invention can be either crystalline, amorphous, or a mixture thereof, it is generally preferred that the drug be present in the formulation in an amorphous form. This would lead to greater ease of dissolution and absorption, resulting in better bioavailability.

Bis(thiohydrazide amides)-containing formulations according to the invention can be lyophilized, and in general be conveniently reconstituted at concentrations greater than about 5 mg/ml (with concentrations greater than about 6 mg/ml preferred, and concentrations greater than about 8 mg/ml being especially preferred).

Another advantage of bis(thiohydrazide amides)-containing formulations according to the invention is their suitability for administration using standard i.v. infusion tubing due to the small size of the particles.

Bis(thiohydrazide amides)-containing formulations according to the invention can be administered employing relatively small volumes for delivery, e.g., typically requiring infusion volumes <200 ml for a therapeutic dose. In addition, infusion can typically be accomplished over a relatively short period of time, e.g., over about 2-3 hrs, delivering doses >about 88-438 mg/m².

As readily recognized by those of skill in the art, invention compositions can be administered over a variety of time-frames. Of course it is recognized that the more quickly a medicament can be delivered to a patient, the less intrusive the procedure will be. Accordingly, it is presently preferred that the administration period is no greater than about 3 hours, about 2 hours preferably about 1 hour, and that the treatment cycle last no greater than about 2 weeks.

In one embodiment the composition is in the form of a lyophilized powder for reconstitution and intravenous administration. When reconstituted with a suitable aqueous medium such as 0.9% sodium chloride injection or 5% dextrose or 5% glucose injection, the composition forms a stable colloidal solution of bis(thiohydrazide amide). The size of the colloidal suspension may range from 20 nm to 8 microns with a preferred range of about 20-400 nm. In one embodiment the compositions of the present invention can be reconstituted in a wide range of concentrations ranging from dilute (0.1 mg/ml) to concentrated (20 mg/ml). This can result in fairly small volumes of administration.

It is known that colloidal nanoparticles or particles <200 nm in size tend to concentrate at the tumor site due to leaky vasculatures. This effect has been described for several liposomal formulations (Papahadjopoulos, et al., 1991; “Sterically Stabilized Liposomes: improvements in pharmacokinetics, and anti-tumor therapeutic efficacy”, Proc. Natl. Acad. Sci. U.S.A. 88, 11460, 1991; Gabizon, A., 1992, “Selective tumor localization and improved therapeutic index of anthracyclines encapsulated in long-circulating liposomes”, Cancer Res., 52, 891, 1992; Dvorak, et al., 1988, “Identification and Characterization of the blood vessels of solid tumors that are leaky to circulating macromolecules”, Am. J. Pathol., 133, 95, 1988; Dunn, et al., 1994, Polystyrene-pol(ethylene glycol) PS-PEG 2000 particles as model systems for site specific drug delivery: The effect of PEG surface density on the in vitro cell interactions and in vivo biodistribution. Pharm, Res., 11:1016-1022 (1994); and Gref, et al, 1994); Biodegradable long-circulating polymeric nanospheres. Science 263:1600-1603 (1994)). It is possible that localized nanoparticles of bis(thiohydrazide amide) at the tumor site may result in slow release of the drug at the tumor site resulting in greater efficacy.

The delivery of bis(thiohydrazide amides) in the form of a microparticulate suspension in general allows some degree of targeting to organs such as the liver, lungs, spleen, lymphatic circulation, and the like, through the use of particles of varying size, and through administration by different routes.

In one embodiment of the present invention, there are provided methods for the treatment of primary tumors in a subject by achieving high local concentration of bis(thiohydrazide amides) at the tumor site, said methods comprising systemically administering bis(thiohydrazide amides) to said subject in a pharmaceutically acceptable formulation as described herein.

In accordance with another embodiment of the present invention, there is provided a method for the preparation of a bis(thiohydrazide amides) for in vivo delivery, said method comprising subjecting a mixture comprising: dispersing agent containing bis(thiohydrazide amides) dispersed therein, and aqueous medium containing biocompatible polymer capable of being crosslinked by disulfide bonds, to sonication conditions for a time sufficient to promote crosslinking of said biocompatible polymer by disulfide bonds.

In accordance with the present invention, the polymer (e.g., a protein) is selectively chemically crosslinked through the formation of disulfide bonds through, for example, the amino acid cysteine that occurs in the natural structure of a number of proteins. A sonication process is used to disperse a dispersing agent containing dissolved or suspended bis(thiohydrazide amides) into an aqueous solution of a biocompatible polymer bearing sulfhydryl or disulfide groups (e.g., albumin) whereby a shell of crosslinked polymer is formed around fine droplets of non-aqueous medium. The sonication process in general produces cavitation in the liquid that causes tremendous local heating and results in the formation of superoxide ions that crosslink the polymer by oxidizing the sulfhydryl residues (and/or disrupting existing disulfide bonds) to form new, crosslinking disulfide bonds.

One feature of the above-described process is in the choice of dispersing agent, specifically with respect to the polarity of the dispersing agent. The formation of a shell about the particles of bis(thiohydrazide amides) involves unfolding and reorientation of the polymer at the interface between the aqueous and non-aqueous phases such that the hydrophilic regions within the polymer are exposed to the aqueous phase while the hydrophobic regions within the polymer are oriented towards the non-aqueous phase. In order to effect unfolding of the polymer, or change the conformation thereof, energy must be supplied to the polymer. The interfacial free energy (interfacial tension) between the two liquid phases (i.e., aqueous and non-aqueous) contributes to changes in polymer conformation at that interface. Thermal energy also contributes to the energy pool required for unfolding and/or change of polymer conformation.

Thermal energy input is a function of such variables as the acoustic power employed in the sonication process, the sonication time, the nature of the material being subjected to sonication, the volume of the material being subjected to sonication, and the like. The acoustic power of sonication processes can vary widely, typically falling in the range of about 1 up to 1000 watts/cm²; with an acoustic power in the range of about 50 up to 200 watts/cm² being a presently preferred range. Similarly, sonication time can vary widely, typically falling in the range of a few seconds up to about 5 minutes. Preferably, sonication time will fall in the range of about 15 up to 60 seconds. Those of skill in the art recognize that the higher the acoustic power applied, the less sonication time is required, and vice versa.

The interfacial free energy is directly proportional to the polarity difference between the two liquids. Thus at a given operating temperature a minimum free energy at the interface between the two liquids is essential to form the desired polymer shell. Thus, if a homologous series of dispersing agents is taken with a gradual change in polarity, e.g., ethyl esters of alkanoic acids, then higher homologues are increasingly nonpolar, i.e., the interfacial tension between these dispersing agents and water increases as the number of carbon atoms in the ester increases. Thus it is found that, although ethyl acetate is water-immiscible (i.e., an ester of a 2 carbon acid), at room temperature (about 20° C.), this dispersing agent alone will not give a significant yield of polymer shell-coated particles. In contrast, a higher ester such as ethyl octanoate (ester of an 8 carbon acid) gives polymer shell-coated particles in high yield. In fact, ethyl heptanoate (ester of a 7 carbon acid) gives a moderate yield while the lower esters (esters of 3, 4, 5, or 6 carbon acids) give poor yield. Thus, at a given temperature, one could set a condition of minimum aqueous-dispersing agent interfacial tension required for formation of high yields of polymer shell-coated particles.

Temperature is another variable that may be manipulated to affect the yield of polymer shell-coated particles. In general the surface tension of a liquid decreases with increasing temperature. The rate of change of surface tension with temperature is often different for different liquids. Thus, for example, the interfacial tension (Δγ) between two liquids may be Δγ₁ at temperature T₁ and Δγ₂ at temperature T₂. If Δγ₁ at T₁ is close to the minimum required to form polymeric shells of the present invention, and if Δγ₂ (at temp. T₂) is greater than Δγ₁ then a change of temperature from T₁ to T₂ will increase the yield of polymeric shells. This, in fact, is observed in the case of ethyl heptanoate, which gives a moderate yield at 20° C. but gives a high yield at 10° C.

Temperature also affects the vapor pressure of the liquids employed. The lower the temperature, the lower the total vapor pressure. The lower the total vapor pressure, the more efficient is the collapse of the cavitation bubble. A more efficient collapse of the sonication bubble correlates with an increased rate of superoxide (HO₂⁻) formation. Increased rate of superoxide formation leads to increased yields of polymeric shells at lower temperatures. As a countervailing consideration, however, the reaction rate for oxidation of sulfhydryl groups (i.e., to form disulfide linkages) by superoxide ions increases with increasing temperature. Thus for a given liquid subjected to sonication conditions, there exists a fairly narrow range of optimum operating temperatures within which a high yield of polymeric shells is obtained.

Thus a combination of two effects, i.e., the change in surface tension with temperature (which directly affects unfolding and/or conformational changes of the polymer) and the change in reaction yield (the reaction being crosslinking of the polymer via formation of disulfide linkages) with temperature dictate the overall conversion or yield of polymer shell-coated particles.

The sonication process described herein may be manipulated to produce polymer shell-coated particles containing bis(thiohydrazide amide) having a range of sizes. Presently preferred particle radii fall in the range of about 0.1 up to about 5 micron. A narrow size distribution in this range is very suitable for intravenous drug delivery. The polymer shell-coated particles are then suspended in an aqueous biocompatible liquid (as described above) prior to administration by suitable means.

Thus, in accordance with the present invention, bis(thiohydrazide amides) contained within polymeric shells are synthesized using high intensity ultrasound. Two non-linear acoustic processes are involved in the formation of stable polymeric shells (i.e., acoustic emulsification and cavitation). First, acoustic emulsification disperses the bis(thiohydrazide amide) into the aqueous protein solution. The dispersion formed is then chemically crosslinked and stabilized by the formation of disulfide bonds. The disulfide bonds are formed from the cysteine residues (in the case where the polymer is a protein such as albumin) that are oxidized by superoxide which is produced via acoustic cavitation.

The resulting suspension is optionally filtered through centricon filters (100 kDa cutoff) and the filtered constructs or microbubbles are resuspended in normal saline or suitable buffer. In general the average diameter of these constructs is approximately 2 microns. Particle size distribution, in general has a mean diameter of about 3 microns. The size range of particles obtained by this technique in general are between 0.1 micron to 20 microns. A preferred size range is 0.5 to 10 microns and the most preferred range is 1 to 5 microns. This size is ideally suited for medical applications, since intravenous or intraarterial injections can be accomplished without risk of small blood vessel blockage and subsequent tissue (ischemia due to oxygen deprivation) damage.

Process for preparing the polymeric shells useful in the formulation of the present invention are described in U.S. Pat. Nos. 5,439,686, 5,498,421, 6,096,331, 6,506,405, 6,537,579, 6,749,868, 6,753,006, 5,665,382, 5,560,933, and 5,916,596 the entire contents of each of which are incorporated herein by reference.

In one embodiment, the present invention, provides methods for the formation of nanoparticles of bis(thiohydrazide amides) by a solvent evaporation technique from an oil-in-water emulsion prepared under conditions of high shear forces (e.g., sonication, high pressure homogenization, or the like), optionally without the use of any conventional surfactants and/or without the use of any polymeric core material to form the matrix of the nanoparticle. Instead, proteins (e.g., human serum albumin) are employed as a stabilizing agent.

The invention further provides a method for the reproducible formation of unusually small nanoparticles (less than 200 nm diameter), which can be sterile-filtered through a 0.22 micron filter. This is achieved by addition of a water soluble solvent (e.g., ethanol) to the organic phase and by carefully selecting the type of organic phase, the phase fraction and the drug concentration in the organic phase. The ability to form nanoparticles of a size that is filterable by 0.22 micron filters is of great importance and significance, since formulations which contain a significant amount of any protein (e.g., albumin), cannot be sterilized by conventional methods such as autoclaving, due to the heat coagulation of the protein.

In one embodiment, surfactant is not be added to the composition in the methods of the present invention. Surfactant can desirably be added to the composition, however, when additional control over solvation of the bis(thiohydrazide amide) is warranted. When used, exemplary surfactants include sodium lauryl sulfate, lecithin, Spans, Tweens (e.g., tween 80, and the like), block copolymers (e.g., pluronics (e.g., pluronic F-68, and the like), tetronics, and the like), and other pharmaceutically acceptable surfactants, and suitable combinations of any two or more thereof.

In one embodiment, foam suppressant is not be added to the composition in the methods of the present invention. Foam suppressant can desirably be added to the composition, however, when additional control over the suppression of foam in the formation of the nanoparticles is warranted. When used, exemplary foam suppressants include silicones, oils, hydrocarbons, alcohols, other compounds which function to suppress foaming in the formation of the nanoparticles, and the like, and suitable combinations of any two or more thereof.

The order in which these components are added to the oil phase and/or the aqueous phase can be varied depending on various conditions, as recognized by those of skill in the art.

Thus, although polymer, and/or surfactant, and/or foam suppressant can optionally be added, the oil phase employed in the preparation of invention compositions typically contains only the bis(thiohydrazide amide) dissolved in solvent, and the aqueous phase employed in the preparation of invention compositions commonly contains only the protein dissolved in aqueous medium.

In one embodiment in the methods of the present invention, an emulsion is formed by homogenization under high pressure and high shear forces of the aqueous and organic phases comprising the polymer and bis(thiohydrazide amide) respectively. Such homogenization is conveniently carried out in a high pressure homogenizer, typically operated at pressures in the range of about 100 up to about 100,000 psi, and preferably in the range of about 2,000 up to 60,000 psi, and can be in a presently preferred range of about 3,000 to about 40,000 psi. In one operational embodiment, such processes can be carried out at a predetermined pressure in the range of about 3,000 psi up to about 30,000 psi. In a presently preferred embodiment, such processes are carried out at pressures in the range of about 6,000 up to 25,000 psi, and even as high as 40,000 psi. The resulting emulsion comprises very small nanodroplets of the nonaqueous solvent (containing the dissolved bis(thiohydrazide amide)) and very small nanodroplets of the protein stabilizing agent. Acceptable methods of homogenization include processes imparting high shear and cavitation such as high pressure homogenization, high shear mixers, sonication, high shear impellers, and the like. Processes imparting shear and cavitation forces accomplish high pressure homogenization by using devices such as sonicators, homogenizers, mixers, impellers, and the like (e.g., devices commercially available from such sources as Heat Systems, Microfluidics, Avestin, Stansted, APV, Gaulin, Rannie, Ross, Silverson, Niro, and the like), and suitable combinations of any two or more thereof.

When high pressure homogenization equipment (e.g., a microfluidizer, and the like) is utilized, the product passes through an interaction chamber or a homogenizing valve which channels the product through narrow orifices with tortuous paths (10 μm-2000 μm nominal diameter) which provides high levels of shear in order to break down particle size. Different interaction chambers or homogenizing valves provide different levels of shearing force and thus break down the particle size to different extents. Interaction chambers and homogenizing valves are chosen based on their ability to reduce the particle size. The product can also be extruded under pressure through membranes or other devices having small pores whose size is in the range from about 0.025 micron to about several (e.g., up to about 200) microns.

Finally, the solvent is evaporated under reduced pressure to yield a colloidal system composed of protein coated nanoparticles of bis(thiohydrazide amide) and protein. As readily recognized by those of skill in the art, a wide variety of methods of evaporation are suitable for use in the practice of the present invention, including using device(s) selected from rotary evaporators, film evaporators, rising film evaporators, falling film evaporators, agitated film evaporators (e.g., Rototherm), concentrators, evaporator/strippers, multistage evaporators, spray driers, lyophilizers, flash evaporators, freeze driers, or combinations of different types of evaporators such as those available from Buchi, LCI, Artisan, Pope, and Niro, or the like, or suitable combinations of any two or more thereof.

Optionally, the colloidal system produced upon evaporation of the solvent can be ultrafiltered for further concentration or to remove small molecules (e.g., organics, salts, contaminants, and the like). As readily recognized by those of skill in the art, this ultrafiltration can be accomplished by a variety of methodologies adaptable to the practice of the present invention, e.g., by using ultrafiltration device(s) such as those commercially available from Sartorius, Millipore, Pall, and the like. This ultrafiltration can be conducted prior to, in between, or after the optional filtration(s) identified in the succeeding paragraph, e.g., prior to conventional filtration, in between the stages of prefiltration and sterile filtration or after sterile filtration.

As a further optional step, the colloidal system produced upon evaporation of the solvent can be conventionally filtered and/or sterilized by filtration through sterilizing filter(s) (e.g., sterilizing filters such as membrane filters, track etched filters, depth filters and the like, and suitable combinations of any two or more thereof). Exemplary sterilizing filters are commercially available from Sartorius, Millipore, Gelman, Pall, Nuclepore, and the like. Where prefiltration is desirable, prefilter(s) can be utilized prior to sterile filtration.

In addition, the entire process of manufacture of the product (e.g., the preparation of the mixture, and/or the formation of the emulsion by homogenization, and/or the formation of the colloidal system by evaporation of the solvent, and/or the ultrafiltration, and/or the sterile filtration, as applicable) can be conducted in a batchwise mode or in a continuous mode or by a combination of batch and continuous processes.

Thus, for example, the homogenizer equipment mentioned above (for example, the microfluidizer) can be operated in a number of different ways, e.g., utilizing batch processes, continuous processes or a combination of batch and continuous processes. For example, this homogenizer equipment can be operated in the recycle mode with continuous recycling until the product meets the required particle size, and/or with discrete cycling (i.e., all of the product is processed for a fixed number of cycles (passes)), and/or in a continuous mode with recycle while removing a fixed percentage of the recycled product continuously. In addition, multiple units of the homogenizer equipment can be connected in series to achieve the desired quality for the product.

Similarly, the evaporator equipment can be operated in batch mode, continuous mode or by a combination of batch and continuous processes. For continuous mode evaporation, the product can be processed once through, or can be recycled continuously through the evaporator until such time as the desired quality of product is attained. For batch mode evaporation, the product may be processed once through the evaporator, provided the desired quality of product is achieved.

Following evaporation of solvent, the liquid suspension may be dried to obtain a powder containing the bis(thiohydrazide amide) and protein. The resulting powder can be redispersed at any convenient time into a suitable aqueous medium such as saline, buffered saline, water, buffered aqueous media, solutions of amino acids, solutions of vitamins, solutions of carbohydrates, or the like, as well as combinations of any two or more thereof, to obtain a suspension that can be administered to mammals. Methods contemplated for obtaining this powder include freeze-drying, spray drying, and the like.

In order to obtain sterile-filterable particles (i.e., particles <200 nm), bis(thiohydrazide amides) are initially dissolved in a substantially water immiscible organic solvent (e.g., a solvent having less than about 5% solubility in water, such as, for example, chloroform, and other suitable solvents and organic solvents as described below) at high concentration, thereby forming an oil phase containing the bis(thiohydrazide amide). The oil phase employed in the process of the present invention generally contains only the bis(thiohydrazide amide) dissolved in solvent.

Next, a water miscible organic solvent (e.g., a solvent having greater than about 10% solubility in water, such as, for example, ethanol) is optionally added to the oil phase at a final concentration in the range of about 1%-99% v/v, more preferably in the range of about 5%-25% v/v of the total organic phase. The water miscible organic solvent can be selected from such solvents as ethyl acetate, ethanol, tetrahydrofuran, dioxane, acetonitrile, acetone, dimethyl sulfoxide, dimethyl formamide, methyl pyrrolidinone, and the like, and other suitable solvents and organic media as described below. Alternatively, when water miscible solvent is to be added, the mixture of water immiscible solvent with the water miscible solvent is prepared first, followed by dissolution of the bis(thiohydrazide amide) in the mixture.

Next, human serum albumin or any other suitable stabilizing agent as described herein is dissolved in aqueous media. This component acts as a stabilizing agent for the formation of stable nanodroplets. Optionally, a sufficient amount of the first organic solvent (i.e., the substantially water immiscible organic solvent discussed above, e.g., chloroform) is dissolved in the aqueous phase to bring it close to the saturation concentration. A separate, measured amount of the total organic phase (which now contains the bis(thiohydrazide amide), the first organic solvent and optionally the second organic solvent) is added to the saturated aqueous phase, so that the phase fraction of the organic phase is between about 0.1%-50% v/v, and more preferably between 1% and 15% v/v.

As discussed above, polymer(s) and/or surfactant(s) and/or foam suppressant(s) need not be added to the mixture, although such surfactant(s) and/or foam suppressant(s) can be added when additional control over the nanoparticle size, and/or additional control over solvation of the bis(thiohydrazide amide), and/or over the suppression of foam in the formation of the nanoparticle, respectively, is desirable.

Next, a mixture composed of micro and nanodroplets is formed by homogenization at low shear forces. This can be accomplished in a variety of ways, as can readily be identified by those of skill in the art, employing, for example, a conventional laboratory homogenizer operated in the range of about 2,000 up to about 15,000 rpm. This is followed by homogenization under high pressure (i.e., in the range of about 100 up to about 100,000 psi, and preferably in the range of about 2,000 up to about 60,000 psi, and can be in a presently preferred range of about 3,000 to about 40,000 psi). In one operational embodiment, such high pressure homogenization can be carried out at a predetermined pressure in the range of about 3,000 psi up to about 30,000 psi. The resulting mixture comprises an aqueous protein solution (e.g., human serum albumin), the water insoluble bis(thiohydrazide amide), and the organic solvent(s). Finally, solvent is rapidly evaporated under vacuum to yield a colloidal dispersion system (bis(thiohydrazide amide) and protein) in the form of extremely small nanoparticles (i.e., particles in the range of about 10 nm-200 nm diameter), and thus can be sterile-filtered, and optionally conventionally filtered and/or ultra-filtered. The preferred size range of the particles is between about 50 nm-170 nm, depending on the formulation and operational parameters.

Colloidal systems prepared in accordance with the present invention may be further converted into powder form by removal of the water therefrom, e.g., by lyophilization at a suitable temperature-time profile. As recognized by those of skill in the art, other conventional modes of water removal (e.g., spray drying) can be adapted to the practice of the present invention. The protein (e.g., human serum albumin) itself acts as a cryoprotectant, and the powder is easily reconstituted by addition of water, saline or buffer, without the need to use such conventional cryoprotectants as mannitol, sucrose, glycine, and the like. While not required, it is of course understood that conventional cryoprotectants may be added to invention formulations if so desired.

The invention further provides a drug delivery system in which part of the molecules of bis(thiohydrazide amides) are bound to the protein (e.g., human serum albumin), and are therefore immediately bioavailable upon administration to a mammal. The other portion of the bis(thiohydrazide amide) is contained within nanoparticles coated by protein. The nanoparticles containing bis(thiohydrazide amides) are present as a substantially pure active component, without dilution by much, if any, polymeric matrix.

A large number of conventional pharmacologically active agents circulate in the blood stream bound to carrier proteins (through hydrophobic or ionic interactions) of which the most common example is serum albumin. Invention methods and compositions produced thereby provide for a bis(thiohydrazide amide) that is “pre-bound” to a protein (through hydrophobic or ionic interactions) prior to administration

In addition, advantage is taken of the capability of human serum albumin to bind bis(thiohydrazide amides), as well as other drugs, which enhances the capability of bis(thiohydrazide amides) to absorb on the surface of the particles. Since albumin is present on the colloidal drug particles (formed upon removal of the organic solvent), formation of a colloidal dispersion which is stable for prolonged periods is facilitated, due to a combination of electrical repulsion and steric stabilization.

In accordance with a further embodiment of the present invention, there is provided a drug delivery system comprising particles of bis(thiohydrazide amide), coated with a protein, wherein said protein coating has free protein associated therewith, wherein a portion of said bis(thiohydrazide amide) is contained within said protein coating and a portion of said bis(thiohydrazide amide) is associated with said free protein. In a specific embodiment, there is provided a drug delivery system comprising particles of bis(thiohydrazide amide), wherein a portion of the bis(thiohydrazide amide) is contained within the protein coating and a portion of the bis(thiohydrazide amide) is bound to protein at a surface of the protein coating. In one embodiment the average diameter of said particles is no greater than about 1 micron.

Suitable solvents utilized in accordance with the methods of the present invention include chloroform, methylene chloride, ethyl acetate, ethanol, tetrahydrofuran, dioxane, acetonitrile, acetone, dimethyl sulfoxide, dimethyl formamide, methylpyrrolidinone, and the like, as well as mixtures of any two or more thereof. Additional solvents contemplated for use in the practice of the present invention include soybean oil, coconut oil, olive oil, safflower oil, cotton seed oil, sesame oil, orange oil, limonene oil, C1-C20 alcohols (e.g., 1-butanol, 2-butanol, 1-pentanol, 3-methyl 1-butanol, and the like), C2-C20 esters (e.g., butyl acetate, isobutyl acetate, isopropyl acetate, n-isopropyl acetate, and the like), C3-C20 ketones, polyethylene glycols, aliphatic hydrocarbons (e.g., heptane, pentane, and the like), aromatic hydrocarbons, halogenated hydrocarbons, and combinations thereof.

Especially preferred combinations of organic media contemplated for use in the practice of the present invention typically have a boiling point of no greater than about 200° C., and include volatile liquids such as dichloromethane, chloroform, ethyl acetate, benzene, and the like (i.e., solvents that have a high degree of solubility for the bis(thiohydrazide amide), and are soluble in the other organic medium employed), along with a higher molecular weight (less volatile) organic medium. When added to the other organic medium, these volatile additives help to drive the solubility of the bis(thiohydrazide amide) into the organic medium. This is desirable since this step is usually time consuming. Following dissolution, the volatile component may be removed by evaporation (optionally under vacuum).

Suitable solvent and/or organic media is typically added at a concentration in the range of about 0.01% (w/v) to about 50% (w/v), as measured in the final mixture prior to evaporation and lyophilization.

Optionally, temperature sensitive materials (e.g., copolymers of polyacrylamides, copolymers of polyalkylene glycols and/or polylactide/glycolides, and the like) which gel at the local site (e.g., localized tumor site, and the like) can be utilized as the dispersing matrix for the invention formulation. In addition, gels could be made of other polysaccharides (e.g., chemically modified hyaluronic acid, and the like) and/or proteins (e.g., albumin, and the like) for controlled release of drugs from nanoparticle formulations.

These matrix-dispersed formulations can be delivered locally by a variety of means of local delivery, as discussed above (e.g., implantation directly into the brain or the peritoneal cavity after surgical removal of the brain tumor or peritoneal-located tumor, respectively, and the like). When temperature sensitive materials are utilized in the formation of this matrix, the invention formulations can be injected in a liquid formulation of the temperature sensitive materials which gels at the tumor site and provides for slow release of the bis(thiohydrazide amide).

The bis(thio-hydrazide amides) employed in methods and compositions or the present invention are represented by Structural Formula I and pharmaceutically acceptable salts and solvates of the compounds represented by Structural Formula I.

In one embodiment, Y in Structural Formula I is a covalent bond, —C(R₅R₆)—, —(CH₂CH₂)—, trans-(CH═CH)—, cis-(CH═CH)— or —(C≡C)— group, preferably —C(R₅R₆)—. R₁-R₄ are as described above for Structural Formula I. R₅ and R₆ are each independently —H, an aliphatic or substituted aliphatic group, or R₅ is —H and R₆ is an optionally substituted aryl group, or, R₅ and R₆, taken together, are an optionally substituted C2-C6 alkylene group. In one embodiment, the compound of Structural Formula I is in the form of a pharmaceutically acceptable salt. In one embodiment, the compound of Structural Formula I is in the form of a pharmaceutically acceptable salt in combination with one or more pharmaceutically acceptable cations. The pharmaceutically acceptable cations are as described in detail below.

In specific embodiments, Y taken together with both >C=Z groups to which it is bonded, is an optionally substituted aromatic group. In this instance, certain bis(thio-hydrazide amides) are represented by Structural Formula II:

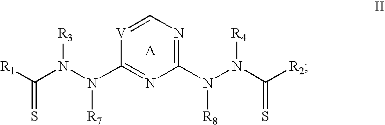

wherein Ring A is substituted or unsubstituted and V is —CH— or —N—. The other variables in Structural Formula II are as described herein for Structural Formula I or IIIa.

In particular embodiments, the bis(thio-hydrazide amides) are represented by Structural Formula IIIa:

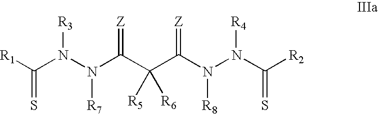

### R₁-R₈ are as described above for Structural Formula I.

In Structural Formulas I-IIIa, R₁ and R₂ are the same or different and/or R₃ and R₄ are the same or different; preferably, R₁ and R₂ are the same and R₃ and R₄ are the same. In Structural Formulas I and IIIa, Z is preferably O. Typically in Structural Formulas I and IIIa, Z is O; R₁ and R₂ are the same; and R₃ and R₄ are the same. More preferably, Z is O; R₁ and R₂ are the same; R₃ and R₄ are the same, and R₇ and R₈ are the same.

In other embodiments, the bis(thio-hydrazide amides) are represented by Structural Formula IIIa: R₁ and R₂ are each an optionally substituted aryl group, preferably an optionally substituted phenyl group; R₃ and R₄ are each an optionally substituted aliphatic group, preferably an alkyl group optionally substituted with —OH, halogen, phenyl, benzyl, pyridyl, or C1-C8 alkoxy and R₆ is —H or methyl, more preferably, methyl or ethyl group optionally substituted with —OH, halogen, phenyl, benzyl, pyridyl, or C1-C8 alkoxy and R₆ is —H or methyl optionally substituted with —OH, halogen or C1-C4 alkoxy; and R₅ and R₆ are as described above, but R₅ is preferably —H and R₆ is preferably —H, an aliphatic or substituted aliphatic group.

Alternatively, R₁ and R₂ are each an optionally substituted aryl group; R₃ and R₄ are each an optionally substituted aliphatic group; R₅ is —H; and R₆ is —H, an aliphatic or substituted aliphatic group. Preferably, R₁ and R₂ are each an optionally substituted aryl group; R₃ and R₄ are each an alkyl group optionally substituted with —OH, halogen, phenyl, benzyl, pyridyl, or C1-C8 alkoxy and R₆ is —H or methyl; and R₅ is —H and R₆ is —H or methyl. Even more preferably, R₁ and R₂ are each an optionally substituted phenyl group, preferably optionally substituted with —OH, halogen, C1-4 alkyl or C1-C4 alkoxy; R₃ and R₄ are each methyl or ethyl optionally substituted with —OH, halogen or C1-C4 alkoxy; and R₅ is —H and R₆ is —H or methyl. Suitable substituents for an aryl group represented by R₁ and R₂ and an aliphatic group represented by R₃, R₄ and R₆ are as described below for aryl and aliphatic groups.

In another embodiment, the bis(thio-hydrazide amides) are represented by Structural Formula IIIa: R₁ and R₂ are each an optionally substituted aliphatic group, preferably a C3-C8 cycloalkyl group optionally substituted with at least one alkyl group, more preferably cyclopropyl or 1-methylcyclopropyl; R₃ and R₄ are as described above for Structural Formula I, preferably both an optionally substituted alkyl group; and R₅ and R₆ are as described above, but R₅ is preferably —H and R₆ is preferably —H, an aliphatic or substituted aliphatic group, more preferably —H or methyl.

Alternatively, the bis(thio-hydrazide amides) are represented by Structural Formula IIIa: R₁ and R₂ are each an optionally substituted aliphatic group; R₃ and R₄ are as described above for Structural Formula I, preferably both an optionally substituted alkyl group; and R₅ is —H and R₆ is —H or an optionally substituted aliphatic group. Preferably, R₁ and R₂ are both a C3-C8 cycloalkyl group optionally substituted with at least one alkyl group; R₃ and R₄ are both as described above for Structural Formula I, preferably an alkyl group; and R₅ is —H and R₆ is —H or an aliphatic or substituted aliphatic group. More preferably, R₁ and R₂ are both a C3-C8 cycloalkyl group optionally substituted with at least one alkyl group; R₃ and R₄ are both an alkyl group optionally substituted with —OH, halogen, phenyl, benzyl, pyridyl, or C1-C8 alkoxy and R₆ is —H or methyl; and R₅ is —H and R₆ is —H or methyl. Even more preferably, R₁ and R₂ are both cyclopropyl or 1-methylcyclopropyl; R₃ and R₄ are both an alkyl group, preferably methyl or ethyl optionally substituted with —OH, halogen or C1-C4 alkoxy; and R₅ is —H and R₆ is —H or methyl.

In particular embodiments, the bis(thio-hydrazide amides) are represented by Structural Formula IIIb:

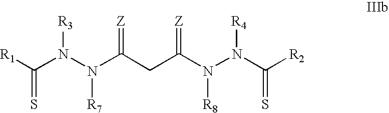

wherein R₁, R₂, R₃, R₄, R₇, R₈, and Z are as defined above for Structural Formula IIIa.

In specific embodiments, the bis(thio-hydrazide amides) are represented by Structural Formula IVa:

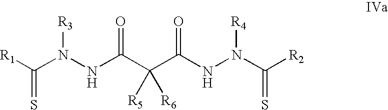

wherein: R₁ and R₂ are both phenyl, R₃ and R₄ are both methyl, and R₅ and R₆ are both —H; R₁ and R₂ are both phenyl, R₃ and R₄ are both ethyl, and R₅ and R₆ are both —H; R₁ and R₂ are both 4-cyanophenyl, R₃ and R₄ are both methyl, R₅ is methyl, and R₆ is —H; R₁ and R₂ are both 4-methoxyphenyl, R₃ and R₄ are both methyl, and R₅ and R₆ are both —H; R₁ and R₂ are both phenyl, R₃ and R₄ are both methyl, R₅ is methyl, and R₆ is —H; R₁ and R₂ are both phenyl, R₃ and R₄ are both ethyl, R₅ is methyl, and R₆ is —H; R₁ and R₂ are both 4-cyanophenyl, R₃ and R₄ are both methyl, and R₅ and R₆ are both —H; R₁ and R₂ are both 2,5-dimethoxyphenyl, R₃ and R₄ are both methyl, and R₅ and R₆ are both —H; R₁ and R₂ are both 2,5-dimethoxyphenyl, R₃ and R₄ are both methyl, R₅ is methyl, and R₆ is —H; R₁ and R₂ are both 3-cyanophenyl, R₃ and R₄ are both methyl, and R₅ and R₆ are both —H; R₁ and R₂ are both 3-fluorophenyl, R₃ and R₄ are both methyl, and R₅ and R₆ are both —H; R₁ and R₂ are both 4-chlorophenyl, R₃ and R₄ are both methyl, R₅ is methyl, and R₆ is —H; R₁ and R₂ are both 2-dimethoxyphenyl, R₃ and R₄ are both methyl, and R₅ and R₆ are both —H; R₁ and R₂ are both 3-methoxyphenyl, R₃ and R₄ are both methyl, and R₅ and R₆ are both —H; R₁ and R₂ are both 2,3-dimethoxyphenyl, R₃ and R₄ are both methyl, and R₅ and R₆ are both —H; R₁ and R₂ are both 2,3-dimethoxyphenyl, R₃ and R₄ are both methyl, R₅ is methyl, and R₆ is —H; R₁ and R₂ are both 2,5-difluorophenyl, R₃ and R₄ are both methyl, and R₅ and R₆ are both —H; R₁ and R₂ are both 2,5-difluorophenyl, R₃ and R₄ are both methyl, R₅ is methyl, and R₆ is —H; R₁ and R₂ are both 2,5-dichlorophenyl, R₃ and R₄ are both methyl, and R₅ and R₆ are both —H; R₁ and R₂ are both 2,5-dimethylphenyl, R₃ and R₄ are both methyl, and R₅ and R₆ are both —H; R₁ and R₂ are both 2,5-dimethoxyphenyl, R₃ and R₄ are both methyl, and R₅ and R₆ are both —H; R₁ and R₂ are both phenyl, R₃ and R₄ are both methyl, and R₅ and R₆ are both —H; R₁ and R₂ are both 2,5-dimethoxyphenyl, R₃ and R₄ are both methyl, R₅ is methyl, and R₆ is —H; R₁ and R₂ are both cyclopropyl, R₃ and R₄ are both methyl, and R₅ and R₆ are both —H; R₁ and R₂ are both cyclopropyl, R₃ and R₄ are both ethyl, and R₅ and R₆ are both —H; R₁ and R₂ are both cyclopropyl, R₃ and R₄ are both methyl, R₅ is methyl, and R₆ is —H; R₁ and R₂ are both 1-methylcyclopropyl, R₃ and R₄ are both methyl, and R₅ and R₆ are both —H; R₁ and R₂ are both 1-methylcyclopropyl, R₃ and R₄ are both methyl, R₅ is methyl and R₆ is —H; R₁ and R₂ are both 1-methylcyclopropyl, R₃ and R₄ are both methyl, R₅ is ethyl, and R₆ is —H; R₁ and R₂ are both 1-methylcyclopropyl, R₃ and R₄ are both methyl, R₅ is n-propyl, and R₆ is —H; R₁ and R₂ are both 1-methylcyclopropyl, R₃ and R₄ are both methyl, and R₅ and R₆ are both methyl; R₁ and R₂ are both 1-methylcyclopropyl, R₃ and R₄ are both ethyl, and R₅ and R₆ are both —H; R₁ and R₂ are both 1-methylcyclopropyl, R₃ is methyl, R₄ is ethyl, and R₅ and R₆ are both —H; R₁ and R₂ are both 2-methylcyclopropyl, R₃ and R₄ are both methyl, and R₅ and R₆ are both —H; R₁ and R₂ are both 2-phenylcyclopropyl, R₃ and R₄ are both methyl, and R₅ and R₆ are both —H; R₁ and R₂ are both 1-phenylcyclopropyl, R₃ and R₄ are both methyl, and R₅ and R₆ are both —H; R₁ and R₂ are both cyclobutyl, R₃ and R₄ are both methyl, and R₅ and R₆ are both —H; R₁ and R₂ are both cyclopentyl, R₃ and R₄ are both methyl, and R₅ and R₆ are both —H; R₁ and R₂ are both cyclohexyl, R₃ and R₄ are both methyl, and R₅ and R₆ are both —H; R₁ and R₂ are both cyclohexyl, R₃ and R₄ are both phenyl, and R₅ and R₆ are both —H; R₁ and R₂ are both methyl, R₃ and R₄ are both methyl, and R₅ and R₆ are both —H; R₁ and R₂ are both methyl, R₃ and R₄ are both t-butyl, and R₅ and R₆ are both —H; R₁ and R₂ are both methyl, R₃ and R₄ are both phenyl, and R₅ and R₆ are both —H; R₁ and R₂ are both t-butyl, R₃ and R₄ are both methyl, and R₅ and R₆ are both —H; R₁ and R₂ are ethyl, R₃ and R₄ are both methyl, and R₅ and R₆ are both —H; or R₁ and R₂ are both n-propyl, R₃ and R₄ are both methyl, and R₅ and R₆ are both —H.

In particular embodiments, the bis(thio-hydrazide amides) are represented by Structural Formula IVb:

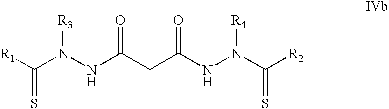

wherein R₁, R₂, R₃, and R₄ are as defined above for Structural Formula IVa.

In specific embodiments, the bis(thio-hydrazide amides) are represented by Structural Formula V:

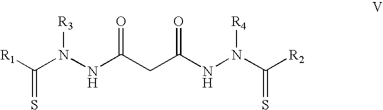

wherein: R₁ and R₂ are both phenyl, and R₃ and R₄ are both o-CH₃-phenyl; R₁ and R₂ are both o-CH₃C(O)O-phenyl, and R₃ and R₄ are phenyl; R₁ and R₂ are both phenyl, and R₃ and R₄ are both methyl; R₁ and R₂ are both phenyl, and R₃ and R₄ are both ethyl; R₁ and R₂ are both phenyl, and R₃ and R₄ are both n-propyl; R₁ and R₂ are both p-cyanophenyl, and R₃ and R₄ are both methyl; R₁ and R₂ are both p-nitro phenyl, and R₃ and R₄ are both methyl; R₁ and R₂ are both 2,5-dimethoxyphenyl, and R₃ and R₄ are both methyl; R₁ and R₂ are both phenyl, and R₃ and R₄ are both n-butyl; R₁ and R₂ are both p-chlorophenyl, and R₃ and R₄ are both methyl; R₁ and R₂ are both 3-nitrophenyl, and R₃ and R₄ are both methyl; R₁ and R₂ are both 3-cyanophenyl, and R₃ and R₄ are both methyl; R₁ and R₂ are both 3-fluorophenyl, and R₃ and R₄ are both methyl; R₁ and R₂ are both 2-furanyl, and R₃ and R₄ are both phenyl; R₁ and R₂ are both 2-methoxyphenyl, and R₃ and R₄ are both methyl; R₁ and R₂ are both 3-methoxyphenyl, and R₃ and R₄ are both methyl; R₁ and R₂ are both 2,3-dimethoxyphenyl, and R₃ and R₄ are both methyl; R₁ and R₂ are both 2-methoxy-5-chlorophenyl, and R₃ and R₄ are both ethyl; R₁ and R₂ are both 2,5-difluorophenyl, and R₃ and R₄ are both methyl; R₁ and R₂ are both 2,5-dichlorophenyl, and R₃ and R₄ are both methyl; R₁ and R₂ are both 2,5-dimethylphenyl, and R₃ and R₄ are both methyl; R₁ and R₂ are both 2-methoxy-5-chlorophenyl, and R₃ and R₄ are both methyl; R₁ and R₂ are both 3,6-dimethoxyphenyl, and R₃ and R₄ are both methyl; R₁ and R₂ are both phenyl, and R₃ and R₄ are both 2-ethylphenyl; R₁ and R₂ are both 2-methyl-5-pyridyl, and R₃ and R₄ are both methyl; or R₁ is phenyl; R₂ is 2,5-dimethoxyphenyl, and R₃ and R₄ are both methyl; R₁ and R₂ are both methyl, and R₃ and R₄ are both p-CF₃-phenyl; R₁ and R₂ are both methyl, and R₃ and R₄ are both o-CH₃-phenyl; R₁ and R₂ are both —(CH₂)₃COOH; and R₃ and R₄ are both phenyl; R₁ and R₂ are both represented by the following structural formula:

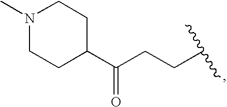

and R₃ and R₄ are both phenyl; R₁ and R₂ are both n-butyl, and R₃ and R₄ are both phenyl; R₁ and R₂ are both n-pentyl, R₃ and R₄ are both phenyl; R₁ and R₂ are both methyl, and R₃ and R₄ are both 2-pyridyl; R₁ and R₂ are both cyclohexyl, and R₃ and R₄ are both phenyl; R₁ and R₂ are both methyl, and R₃ and R₄ are both 2-ethylphenyl; R₁ and R₂ are both methyl, and R₃ and R₄ are both 2,6-dichlorophenyl; R₁-R₄ are all methyl; R₁ and R₂ are both methyl, and R₃ and R₄ are both t-butyl; R₁ and R₂ are both ethyl, and R₃ and R₄ are both methyl; R₁ and R₂ are both t-butyl, and R₃ and R₄ are both methyl; R₁ and R₂ are both cyclopropyl, and R₃ and R₄ are both methyl; R₁ and R₂ are both cyclopropyl, and R₃ and R₄ are both ethyl; R₁ and R₂ are both 1-methylcyclopropyl, and R₃ and R₄ are both methyl; R₁ and R₂ are both 2-methylcyclopropyl, and R₃ and R₄ are both methyl; R₁ and R₂ are both 1-phenylcyclopropyl, and R₃ and R₄ are both methyl; R₁ and R₂ are both 2-phenylcyclopropyl, and R₃ and R₄ are both methyl; R₁ and R₂ are both cyclobutyl, and R₃ and R₄ are both methyl; R₁ and R₂ are both cyclopentyl, and R₃ and R₄ are both methyl; R₁ is cyclopropyl, R₂ is phenyl, and R₃ and R₄ are both methyl.

Preferred examples of bis(thio-hydrazide amides) include Compounds (1)-(18) and pharmaceutically acceptable salts and solvates thereof:

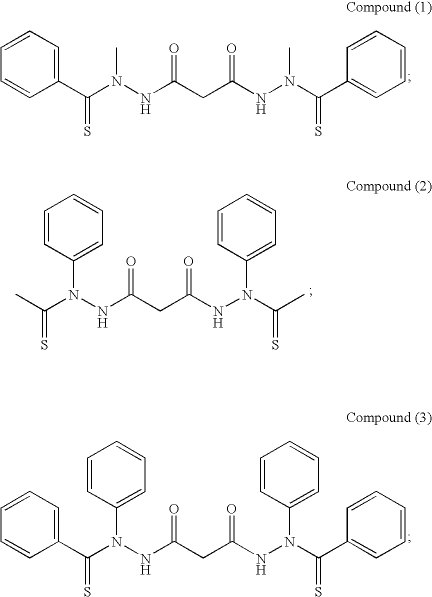

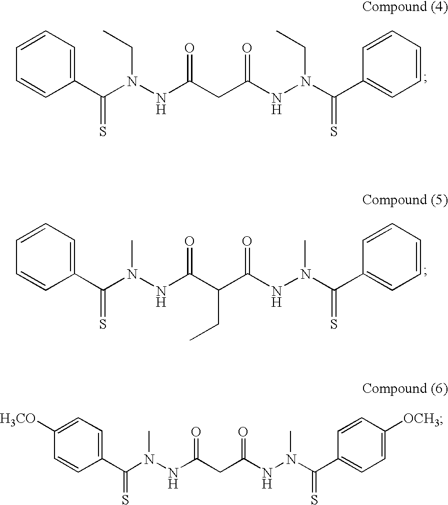

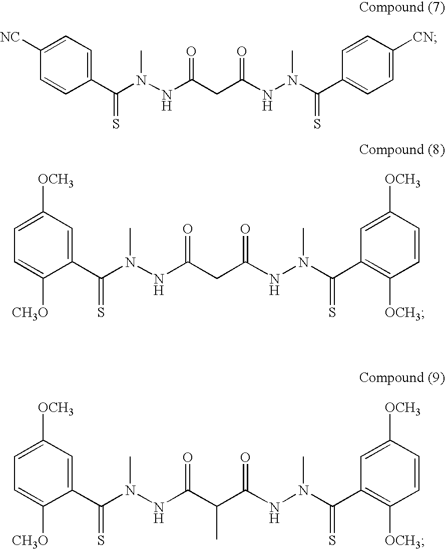

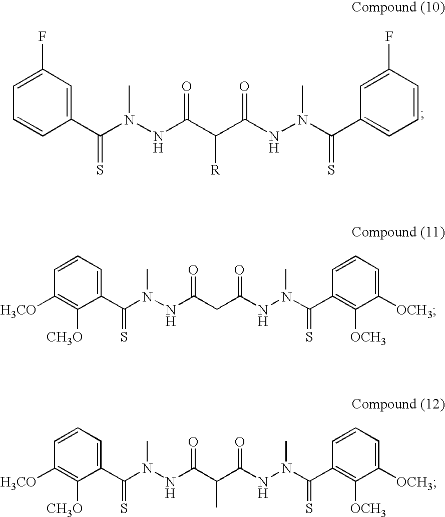

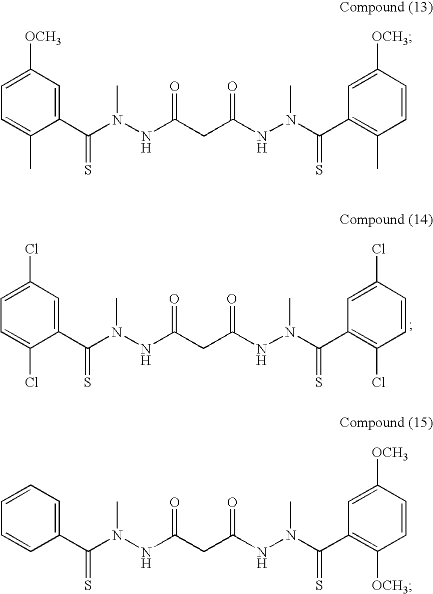

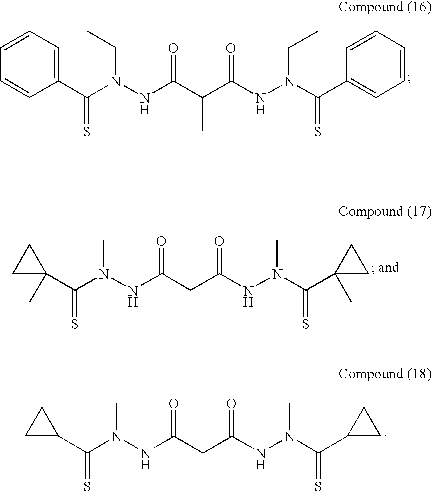

As used herein, the term “bis(thio-hydrazide amide)” and references to the Structural Formulas of this invention also include pharmaceutically acceptable salts and solvates of these compounds and Structural Formulas. Examples of acceptable salts and solvates are described in US Publication No.: 20060135595 and U.S. patent application Ser. No. 11/432,307 filed 11 May 2006, titled Synthesis Of Bis(Thio-Hydrazide Amide) Salts, the entire contents of each of which are incorporated herein by reference.

These compounds can have one or more sufficiently acidic proton that can react with a suitable organic or inorganic base to form a base addition salt. Base addition salts include those derived from inorganic bases, such as ammonium or alkali or alkaline earth metal hydroxides, carbonates, bicarbonates, and the like, and organic bases such as alkoxides, alkyl amides, alkyl and aryl amines, and the like. Such bases useful in preparing the salts of this invention thus include sodium hydroxide, potassium hydroxide, ammonium hydroxide, potassium carbonate, and the like.

For example, pharmaceutically acceptable salts of bis(thio-hydrazide) amides employed herein (e.g., those represented by Structural Formulas I-VI, Compounds 1-18) are those formed by the reaction of the compound with one equivalent of a suitable base to form a monovalent salt (i.e., the compound has single negative charge that is balanced by a pharmaceutically acceptable counter cation, e.g., a monovalent cation) or with two equivalents of a suitable base to form a divalent salt (e.g., the compound has a two-electron negative charge that is balanced by two pharmaceutically acceptable counter cations, e.g., two pharmaceutically acceptable monovalent cations or a single pharmaceutically acceptable divalent cation). Divalent salts of the bis(thio-hydrazide amides) are preferred. “Pharmaceutically acceptable” means that the cation is suitable for administration to a subject. Examples include Li⁺, Na⁺, K⁺, Mg²⁺, Ca²⁺ and NR₄⁺, wherein each R is independently hydrogen, an optionally substituted aliphatic group (e.g., a hydroxyalkyl group, aminoalkyl group or ammoniumalkyl group) or optionally substituted aryl group, or two R groups, taken together, form an optionally substituted non-aromatic heterocyclic ring optionally fused to an aromatic ring. Generally, the pharmaceutically acceptable cation is Li⁺, Na⁺, K⁺, NH₃(C₂H₅OH)⁺ or N(CH₃)₃(C₂H₅OH)⁺, and more typically, the salt is a disodium or dipotassium salt, preferably the disodium salt.

Bis(thio-hydrazide) amides employed herein having a sufficiently basic group, such as an amine can react with an organic or inorganic acid to form an acid addition salt. Acids commonly employed to form acid addition salts from compounds with basic groups are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like. Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

Salts of the disclosed bis(thiohydrazide amides) may have tautomeric forms. By way of example, one tautomeric form for the disalt is:

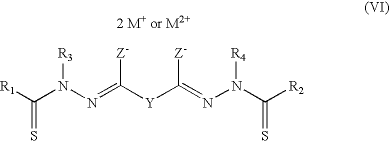

Y is a covalent bond or a substituted or unsubstituted straight chained hydrocarbyl group. R₁-R₄ are independently —H, an aliphatic group, a substituted aliphatic group, an aryl group or a substituted aryl group, or R₁ and R₃ taken together with the carbon and nitrogen atoms to which they are bonded, and/or R₂ and R₄ taken together with the carbon and nitrogen atoms to which they are bonded, form a non-aromatic heterocyclic ring optionally fused to an aromatic ring. Z is —O or —S. M⁺ is a pharmaceutically acceptable monovalent cation and M²⁺ is a pharmaceutically acceptable divalent cation.

In one embodiment, the variables for Structural Formula (VI) are defined below:

M⁺ is a pharmaceutically acceptable monovalent cation. M²⁺ is a pharmaceutically acceptable divalent cation. “Pharmaceutically acceptable” means that the cation is suitable for administration to a subject. Examples of M⁺ or M²⁺ include Li⁺, Na⁺, K⁺, Mg²⁺, Ca²⁺, Zn²⁺, and NR₄⁺, wherein each R is independently hydrogen, a substituted or unsubstituted aliphatic group (e.g., a hydroxyalkyl group, aminoalkyl group or ammoniumalkyl group) or substituted or unsubstituted aryl group, or two R groups, taken together, form a substituted or unsubstituted non-aromatic heterocyclic ring optionally fused to an aromatic ring. Preferably, the pharmaceutically acceptable cation is Li⁺, Na⁺, K⁺, NH₃(C₂H₅OH)⁺, N(CH₃)₃(C₂H₅OH)⁺, arginine or lysine. More preferably, the pharmaceutically acceptable cation is Na⁺ or K⁺. Na⁺ is even more preferred.

Exemplary tautomeric forms of the disalt compounds represented by Structural Formula (VI) wherein Y is —CH₂— are shown below:

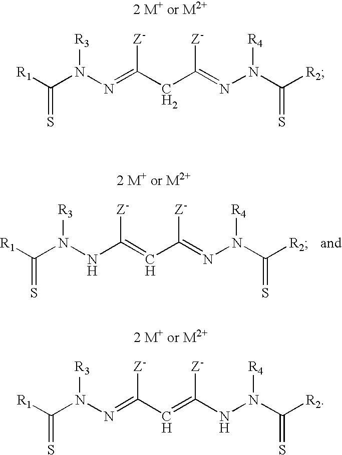

Representative tautomeric structures of the disalt of Compound (1) are shown below:

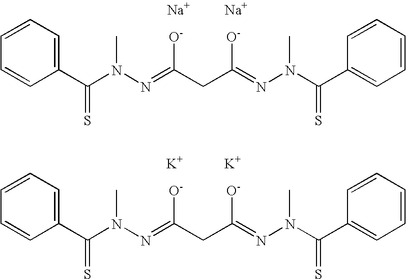

Preferred examples of bis(thio-hydrazide amide) disalts of the present invention are the following:

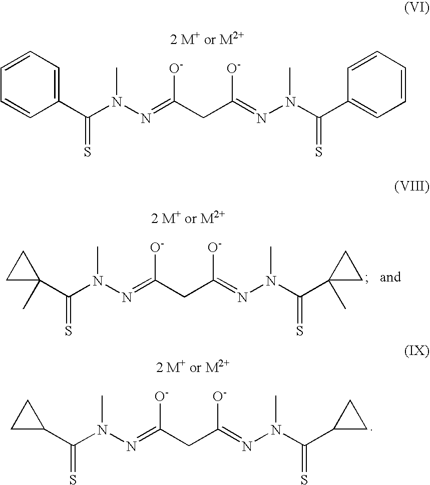

### 2 M⁺ and M²⁺ are as described above for Structural Formula (VI). Preferably, the pharmaceutically acceptable cation is 2 M⁺, wherein M⁺ is Li⁺, Na⁺, K⁺, NH₃(C₂H₅OH)⁺ or N(CH₃)₃(C₂H₅OH)⁺. More preferably, M⁺ is Na⁺ or K⁺. Even more preferably, M⁺ is Na⁺.

It is to be understood when one tautomeric form of a disclosed compound is depicted structurally, other tautomeric forms are also encompassed.

Certain compounds of the invention may be obtained as different stereoisomers (e.g., diastereomers and enantiomers). The invention includes all isomeric forms and racemic mixtures of the disclosed compounds and methods of treating a subject with both pure isomers and mixtures thereof, including racemic mixtures. Stereoisomers can be separated and isolated using any suitable method, such as chromatography.

An “alkyl group” is saturated straight or branched chain linear or cyclic hydrocarbon group. Typically, a straight chained or branched alkyl group has from 1 to about 20 carbon atoms, preferably from 1 to about 10, and a cyclic alkyl group has from 3 to about 10 carbon atoms, preferably from 3 to about 8. An alkyl group is preferably a straight chained or branched alkyl group, e.g., methyl, ethyl, n-propyl, iso-propyl, n-butyl, sec-butyl, tert-butyl, pentyl, hexyl, pentyl or octyl, or a cycloalkyl group with 3 to about 8 carbon atoms. A C1-C8 straight chained or branched alkyl group or a C3-C8 cyclic alkyl group is also referred to as a “lower alkyl” group. Suitable substitutents for an alkyl group are those which do not substantially interfere with the anti-cancer activity of the disclosed compounds. Suitable substituents are as described below for aliphatic groups. Preferred substituents on alkyl groups include, —OH, —NH₂, —NO₂, —CN, —COOH, halogen, aryl, C1-C8 alkoxy, C1-C8 haloalkoxy and —CO(C1-C8 alkyl). More preferred substituents on alkyl groups include —OH, halogen, phenyl, benzyl, pyridyl, and C1-C8 alkoxy. More preferred substituents on alkyl groups include —OH, halogen, and C1-C4 alkoxy.

A “straight chained hydrocarbyl group” is an alkylene group, i.e., —(CH₂)_{y}—, with one or more (preferably one) internal methylene groups optionally replaced with a linkage group. y is a positive integer (e.g., between 1 and 10), preferably between 1 and 6 and more preferably 1 or 2. A “linkage group” refers to a functional group which replaces a methylene in a straight chained hydrocarbyl. Examples of suitable linkage groups include a ketone (—C(O)—), alkene, alkyne, phenylene, ether (—O—), thioether (—S—), or amine (—N(R^{a})—), wherein R^{a} is defined below. A preferred linkage group is —C(R₅R₆)—, wherein R₅ and R₆ are defined above. Suitable substitutents for an alkylene group and a hydrocarbyl group are those which do not substantially interfere with the anti-cancer activity of the disclosed compounds. R₅ and R₆ are preferred substituents for an alkylene or hydrocarbyl group represented by Y.

An aliphatic group is a straight chained, branched or cyclic non-aromatic hydrocarbon which is completely saturated or which contains one or more units of unsaturation. Typically, a straight chained or branched aliphatic group has from 1 to about 20 carbon atoms, preferably from 1 to about 10, and a cyclic aliphatic group has from 3 to about 10 carbon atoms, preferably from 3 to about 8. An aliphatic group is preferably a straight chained or branched alkyl group, e.g., methyl, ethyl, n-propyl, iso-propyl, n-butyl, sec-butyl, tert-butyl, pentyl, hexyl, pentyl or octyl, or a cycloalkyl group with 3 to about 8 carbon atoms. A C1-C8 straight chained or branched alkyl group or a C3-C8 cyclic alkyl group is also referred to as a “lower alkyl” group.

The term “aromatic group” may be used interchangeably with “aryl,” “aryl ring,” “aromatic ring,” “aryl group” and “aromatic group.” Aromatic groups include carbocyclic aromatic groups such as phenyl, naphthyl, and anthracyl, and heteroaryl groups such as imidazolyl, thienyl, furanyl, pyridyl, pyrimidyl, pyranyl, pyrazolyl, pyrroyl, pyrazinyl, thiazole, oxazolyl, and tetrazole. The term “heteroaryl group” may be used interchangeably with “heteroaryl,” “heteroaryl ring,” “heteroaromatic ring” and “heteroaromatic group.” Heteroaryl groups are aromatic groups that comprise one or more heteroatom, such as sulfur, oxygen and nitrogen, in the ring structure. Preferably, heteroaryl groups comprise from one to four heteroatoms.

Aromatic groups also include fused polycyclic aromatic ring systems in which a carbocyclic aromatic ring or heteroaryl ring is fused to one or more other heteroaryl rings. Examples include benzothienyl, benzofuranyl, indolyl, quinolinyl, benzothiazole, benzooxazole, benzimidazole, quinolinyl, isoquinolinyl and isoindolyl.

Non-aromatic heterocyclic rings are non-aromatic rings which include one or more heteroatoms such as nitrogen, oxygen or sulfur in the ring. The ring can be five, six, seven or eight-membered. Preferably, heterocyclic groups comprise from one to about four heteroatoms. Examples include tetrahydrofuranyl, tetrahydrothiophenyl, morpholino, thiomorpholino, pyrrolidinyl, piperazinyl, piperidinyl, and thiazolidinyl.

Suitable substituents on an aliphatic group (including an alkylene group), non-aromatic heterocyclic group, benzylic or aryl group (carbocyclic and heteroaryl) are those which do not substantially interfere with the anti-cancer activity of the disclosed compounds. A substituent substantially interferes with anti-cancer activity when the anti-cancer activity is reduced by more than about 50% in a compound with the substituent compared with a compound without the substituent. Examples of suitable substituents include —R^{a}, —OH, —Br, —Cl, —I, —F, —OR^{a}, —O—COR^{a}, —COR^{a}, —CN, —NO₂, —COOH, —SO₃H, —NH₂, —NHR^{a}, —N(R^{a}R^{b}), —COOR^{a}, —CHO, —CONH₂, —CONHR^{a}, —CON(R^{a}R^{b}), —NHCOR^{a}, —NR^{c}COR^{a}, —NHCONH₂, —NHCONR^{a}H, —NHCON(R^{a}R^{b}), —NR^{c}CONH₂, —NR^{c}CONR^{a}H, —NR^{c}CON(R^{a}R^{b}), —C(═NH)—NH₂, —C(═NH)—NHR^{a}, —C(═NH)—N(R^{a}R^{b}), —C(═NR^{c})—NH₂, —C(═NR^{c})—NHR^{a}, —C(═NR^{c})—N(R^{a}R^{b}), —NH—C(═NH)—NH₂, —NH—C(═NH)—NHR^{a}, —NH—C(═NH)—N(R^{a}R^{b}), —NH—C(═NR^{c})—NH₂, —NH—C(═NR^{c})—NHR^{a}, —NH—C(═NR^{c})—N(R^{a}R^{b}), —NR^{d}H—C(═NH)—NH₂, —NR^{d}—C(═NH)—NHR^{a}, —NR^{d}—C(═NH)—N(R^{a}R^{b}), —NR^{d}—C(═NR^{c})—NH₂, —NR^{d}—C(═NR^{c})—NHR^{a}, —NR^{d}—C(═NR^{c})—N(R^{a}R^{b}), —NHNH₂, —NHNHR^{a}, —NHR^{a}R^{b}, —SO₂NH₂, —SO₂NHR^{a}, —SO₂NR^{a}R^{b}, —CH═CHR^{a}, —CH═CR^{a}R^{b}, —CR^{c}═CR^{a}R^{b}, —CR^{c}═CHR^{a}, —CR^{c}═CR^{a}R^{b}, —CCR^{a}, —SH, —SR^{a}, —S(O)R^{a}, —S(O)₂R^{a}.

R^{a}-R^{d} are each independently an alkyl group, aromatic group, non-aromatic heterocyclic group or —N(R^{a}R^{b}), taken together, form a non-aromatic heterocyclic group. The alkyl, aromatic and non-aromatic heterocyclic group represented by R^{a}-R^{d} and the non-aromatic heterocyclic group represented by —N(R^{a}R^{b}) are each optionally and independently substituted with one or more groups represented by R^{#}. Preferably R^{a}-R^{d} are unsubstituted.

R^{#} is R⁺, —OR⁺, —O(haloalkyl), —SR⁺, —NO₂, —CN, —NCS, —N(R⁺)₂, —NHCO₂R⁺, —NHC(O)R⁺, —NHNHC(O)R⁺, —NHC(O)N(R⁺)₂, —NHNHC(O)N(R⁺)₂, —NHNHCO₂R⁺, —C(O)C(O)R⁺, —C(O)CH₂C(O)R⁺, —CO₂R⁺, —C(O)R⁺, —C(O)N(R⁺)₂, —OC(O)R⁺, —OC(O)N(R⁺)₂, —S(O)₂R⁺, —SO₂N(R⁺)₂, —S(O)R⁺, —NHSO₂N(R⁺)₂, —NHSO₂R⁺, —C(═S)N(R⁺)₂, or —C(═NH)—N(R⁺)₂.

R⁺ is —H, a C1-C4 alkyl group, a monocyclic heteroaryl group, a non-aromatic heterocyclic group or a phenyl group optionally substituted with alkyl, haloalkyl, alkoxy, haloalkoxy, halo, —CN, —NO₂, amine, alkylamine or dialkylamine. Preferably R⁺ is unsubstituted. Optionally, the group —N(R⁺)₂ is a non-aromatic heterocyclic group, provided that non-aromatic heterocyclic groups represented by R⁺ and —N(R⁺)₂ that comprise a secondary ring amine are optionally acylated or alkylated.

Preferred substituents for a phenyl group, including phenyl groups represented by R₁-R₄, include C1-C4 alkyl, C1-C4 alkoxy, C1-C4 haloalkyl, C1-C4 haloalkoxy, phenyl, benzyl, pyridyl, —OH, —NH₂, —F, —Cl, —Br, —I, —NO₂ or —CN. More preferred for a phenyl group, including phenyl groups represented by R₁-R₄, include R₁ and R₂ are optionally substituted with —OH, —CN, halogen, C1-4 alkyl or C1-C4 alkoxy

Preferred substituents for a cycloalkyl group, including cycloalkyl groups represented by R₁ and R₂, are alkyl groups, such as a methyl or ethyl group.

In a particular embodiment, the present invention is a method of treating a subject with cancer comprising administering to the subject an effective amount of a bis(thiohydrazide amide) encapsulated in a polymeric shell as described herein.

Cancers which can be treated by the compositions and methods of the present invention include, but are not limited to, human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, anal carcinoma, esophageal cancer, gastric cancer, hepatocellular cancer, bladder cancer, endometrial cancer, pancreatic cancer, breast cancer, ovarian cancer, prostate cancer, stomach cancer, atrial myxomas, squamous cell carcinoma, basal cell carcinoma, adenocarcinoma, sweat gland carcinoma, sebaceous gland carcinoma, thyroid and parathyroid neoplasms, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, non-small-cell lung cancer, bladder carcinoma, epithelial carcinoma, glioma, pituitary neoplasms, astrocytoma, medulloblastoma, craniopharyngioma, ependymoma, pinealoma, hemangioblastoma, acoustic neuroma, schwannomas, oligodendroglioma, meningioma, spinal cord tumors, melanoma, neuroblastoma, pheochromocytoma, Types 1-3 endocrine neoplasia, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrobm's macroglobulinemia, and heavy chain disease.

Other examples of leukemias include acute and/or chronic leukemias, e.g., lymphocytic leukemia (e.g., as exemplified by the p388 (murine) cell line), large granular lymphocytic leukemia, and lymphoblastic leukemia; T-cell leukemias, e.g., T-cell leukemia (e.g., as exemplified by the CEM, Jurkat, and HSB-2 (acute), YAC-1 (murine) cell lines), T-lymphocytic leukemia, and T-lymphoblastic leukemia; B cell leukemia (e.g., as exemplified by the SB (acute) cell line), and B-lymphocytic leukemia; mixed cell leukemias, e.g., B and T cell leukemia and B and T lymphocytic leukemia; myeloid leukemias, e.g., granulocytic leukemia, myelocytic leukemia (e.g., as exemplified by the HL-60 (promyelocyte) cell line), and myelogenous leukemia (e.g., as exemplified by the K562 (chronic) cell line); neutrophilic leukemia; eosinophilic leukemia; monocytic leukemia (e.g., as exemplified by the THP-1 (acute) cell line); myelomonocytic leukemia; Naegeli-type myeloid leukemia; and nonlymphocytic leukemia. Other examples of leukemias are described in Chapter 60 of The Chemotherapy Sourcebook, Michael C. Perry Ed., Williams & Williams (1992) and Section 36 of Holland Frie Cancer Medicine 5th Ed., Bast et al. Eds., B. C. Decker Inc. (2000). The entire teachings of the preceding references are incorporated herein by reference.

In one embodiment, the methods of the present invention include treating cancers including, but not limited to, non-solid tumors such as multiple myeloma, T-leukemia (e.g., as exemplified by Jurkat and CEM cell lines); B-leukemia (e.g., as exemplified by the SB cell line); promyelocytes (e.g., as exemplified by the HL-60 cell line); uterine sarcoma (e.g., as exemplified by the MES-SA cell line); monocytic leukemia (e.g., as exemplified by the THP-1 (acute) cell line); and lymphoma (e.g., as exemplified by the U937 cell line).

In particular, renal cell carcinoma and melanoma are treated with the disclosed methods. In a particular embodiment, the disclosed method involves treating a subject with melanoma.

Melanoma, can be divided into five main subgroups:

i) Congenital Nevus: which is congenital and not malignant.

ii) Lentigo Maligna (Hutchinsons Freckle): which is a form of melanoma more common among the elderly population. These lesions may grow for years as an in-situ tumor before developing the more aggressive vertical growth phase. This type of melanoma is found most often in the damaged skin on the face, ears, arms, and upper trunk.

iii) Superficial Spreading Malignant Melanoma: is generally the most common form accounting for approximately 65% of diagnosed melanoma. The cancer presumably begins at one focus in the skin at the dermo-epidermal junction. It initially grows in a horizontal plane, along, just above and below the dermo-epidermal junction. This is referred to as the “radial” growth phase of melanoma and is clinically macular or only slightly elevated.

This melanoma travels along the top layer of the skin for a fairly long time before penetrating more deeply. The melanoma can be seen almost anywhere on the body, but is most likely to occur on the trunk in men, the legs in women, and the upper back in both. This type of melanoma is mainly found in the younger population.

iv) Acral Lentiginous Malignant Melanoma: as with superficial spreading malignant melanoma, acral lentiginous malignant melanoma also spreads superficially before penetrating more deeply. It is quite different from the others, though, as it usually appears as a black or brown discoloration under the nails or on the soles of the feet or palms of the hands. This type of melanoma is the most common melanoma in African-Americans and Asians, and the least common among Caucasians.

v) Nodular Malignant Melanoma: is a much less common form of melanoma. Unlike the other types, nodular melanoma, is usually invasive at the time it is first diagnosed. The malignancy is recognized when it becomes a bump. In this tumor, there is presumably no horizontal growth phase. The depth of the lesion appears to correlate with the prognosis of the subject, and nodular melanoma is less often amenable to definitive treatment than is the superficial spreading variety.

The methods of the present invention encompass treating all of the subgroups of melanoma defined above.

Melanoma can further be divided into four different stages, which are divided based on the progression of the disease:

Stage I

Cancer is found in the outer layer of the skin (epidermis) and/or the upper part of the inner layer of skin (dermis), but it has not spread to nearby lymph nodes. The tumor is less than 1.5 millimeters ( 1/16 of an inch) thick.

Stage II

The tumor is 1.5 millimeters to 4 millimeters (less than ⅙ of an inch) thick. It has spread to the lower part of the inner layer of skin (dermis), but not into the tissue below the skin or into nearby lymph nodes.

Stage III

Any of the following mean that the tumor is stage III:

The tumor is more than 4 millimeters (approximately ⅙ of an inch) thick.

The tumor has spread to the body tissue below the skin.

There are additional tumor growths within one inch of the original tumor (satellite tumors).

The tumor has spread to nearby lymph nodes or there are additional tumor growths (satellite tumors) between the original tumor and the lymph nodes in the area

Stage IV

The tumor has spread to other organs or to lymph nodes far away from the original tumor.

In another particular embodiment, the disclosed method involves treating a subject with renal cell carcinoma.

Renal cell carcinoma is the most common type of kidney cancer. It accounts for more than 90% of malignant kidney tumors. Renal cell carcinoma begins small and grows larger over time. Although renal cell carcinoma usually grows as a single mass within the kidney, a kidney may contain more than 1 tumor. Sometimes tumors may be found in both kidneys at the same time. Some renal cell carcinomas are noticed only after they have become quite large; most are found before they metastasize to other organs through the bloodstream or lymph vessels. Like most cancers, renal cell carcinoma is difficult to treat once it has metastasized.

There are five main types of renal cell carcinoma: clear cell, papillary, chromophobe, collecting duct, and “unclassified.”

When viewed under a microscope, the individual cells that make up clear cell renal cell carcinoma appear very pale or clear. This is the most common form of renal cell carcinoma. About 80% of people with renal cell carcinoma have this kind of cancer.

Papillary renal cell carcinoma is the second most common type—about 10% to 15% of people have this kind. These cancers form little finger-like projections (called papillae) in some, if not most, of the tumor. Some doctors call these cancers chromophilic because the cells take up certain dyes used in preparing the tissue to be viewed under the microscope, causing them to appear pink.

Chromophobe renal carcinoma is the third most common type—accounting for about 5% of cases. The cells of these cancers are also pale, like the clear cells, but are much larger and have certain other features that can be recognized.

The fourth type, collecting duct renal carcinoma, is very rare. The major feature is that the cancer cells can form irregular tubes.

About 5% of renal cancers are unclassified because their appearance does not fit into any of the other categories.

Renal cell cancers are usually divided into four stages. The stage describes the cancer's size and how far it has spread beyond the kidney.

The Stage are generally defined below:

Stage I

The tumor is 7 cm or smaller and limited to the kidney. There is no spread to lymph nodes or distant organs.

Stage II:

The tumor is larger than 7 cm but is still limited to the kidney. There is no spread to lymph nodes or distant organs.

Stage III:

This includes:

any tumor that has spread to 1 nearby lymph node but not to more than 1 lymph node or other organs; and/or

tumors that have not spread to lymph nodes or distant organs but have spread to the adrenal glands, to fatty tissue around the kidney, and/or have grown into the large vein (vena cava) leading from the kidney to the heart.

Stage IV:

This includes:

any cancers that have spread directly through the fatty tissue and beyond Gerota fascia, the fibrous tissue that surrounds the kidney; and/or

any cancer that has spread to more than 1 lymph node near the kidney, or to any lymph node distant from the kidney, or to any distant organs such as the lungs, bone, or brain.

The disclosed methods include treating all five types of renal cell carcinoma in all four stages of disease progression as defined immediately above.

The first line treatment for renal cell carcinoma, when detected at an early stage, is often to surgically remove the cancer, for example, by radial nephrectomy. However, in many cases, as many as 20 or 30% of subjects develop metastatic (Stage III or IV) disease. For those subjects with metastatic (Stage III and IV) renal cell carcinoma, the prognosis is bleak.

Treatment of cancers as described above with bis(thiohydrazide amides) are describe din more detail in U.S. provisional Application Nos. 60/839,113, 60/838,986, and 60/838,977, the entire contents of each of which are incorporated herein by reference.

In another embodiment, the disclosed method involves treating subjects whose cancer has become “multi-drug resistant”.

In a particular embodiment, the present invention is a method of treating a subject with cancer comprising administering to the subject an effective amount of a bis(thiohydrazide amide) and an effective amount one or more anti-cancer agents wherein the bis(thiohydrazide amide) is substantially or completely encased in a polymeric shell. In a particular embodiment, the present invention is a method of treating a subject with cancer comprising administering to the subject an effective amount of a bis(thiohydrazide amide) and an effective amount one or more anti-cancer agents wherein the bis(thiohydrazide amide) and the anti-cancer agent are substantially or completely encased in a polymeric shell. In a particular embodiment, the present invention is a method of treating a subject with cancer comprising administering to the subject an effective amount of a bis(thiohydrazide amide) and an effective amount one or more anti-cancer agents wherein the bis(thiohydrazide amide) is substantially or completely encased in a polymeric shell and the anticancer agent is substantially or completely encased within a separate polymeric shell, wherein the polymers shells can be made from the same or different biocompatible polymers as described herein.

Examples of anti-cancer agents/drugs are described below.

In one embodiment the anti-cancer agents/drug is, for example, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer agents/drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; ocreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovorin.

Agents that can be used in the methods of the invention in combination with the bis(thiohydrazide amides) disclosed herein, include but are not limited to, alkylating agents, antimetabolites, natural products, or hormones. Examples of alkylating agents useful in the methods of the invention include but are not limited to, nitrogen mustards (e.g., mechlorethamine, cyclophosphamide, chlorambucil, melphalan, etc.), ethylenimine and methylmelamines (e.g., hexamethylmelamine, thiotepa), alkyl sulfonates (e.g., busulfan), nitrosoureas (e.g., carmustine, lomustine, semustine, streptozocin, etc.), or triazenes (decarbazine, etc.). Examples of antimetabolites useful in the methods of the invention include but are not limited to folic acid analog (e.g., methotrexate), or pyrimidine analogs (e.g., fluorouracil, floxuridine, Cytarabine), purine analogs (e.g., mercaptopurine, thioguanine, pentostatin). Examples of natural products useful in the methods of the invention include but are not limited to vinca alkaloids (e.g., vinblastin, vincristine), epipodophyllotoxins (e.g., etoposide, teniposide), antibiotics (e.g., actinomycin D, daunorubicin, doxorubicin, bleomycin, plicamycin, mitomycin) or enzymes (e.g., L-asparaginase). Examples of hormones and antagonists useful for the treatment or prevention of cancer in the methods of the invention include but are not limited to adrenocorticosteroids (e.g., prednisone), progestins (e.g., hydroxyprogesterone caproate, megestrol acetate, medroxyprogesterone acetate), estrogens (e.g., diethylstilbestrol, ethinyl estradiol), antiestrogen (e.g., tamoxifen), androgens (e.g., testosterone propionate, fluoxymesterone), antiandrogen (e.g., flutamide), gonadotropin releasing hormone analog (e.g., leuprolide). Other agents that can be used in the methods of the invention for the treatment or prevention of cancer include platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide).

Preferably, the anti-cancer agent/drug is an agent that stabilizes microtubules. As used herein, a “microtubulin stabilizer” means an anti-cancer agent/drug which acts by arresting cells in the G2-M phases due to stabilization of microtubules. Examples of microtubulin stabilizers include paclitaxel and TAXOL® analogues. Additional examples of microtubulin stabilizers included without limitation the following marketed drugs and drugs in development: Discodermolide (also known as NVP-XX-A-296); Epothilones (such as Epothilone A, Epothilone B, Epothilone C (also known as desoxyepothilone A or dEpoA); Epothilone D (also referred to as KOS-862, dEpoB, and desoxyepothilone B); Epothilone E; Epothilone F; Epothilone B N-oxide; Epothilone A N-oxide; 16-aza-epothilone B; 21-aminoepothilone B (also known as BMS-310705); 21-hydroxyepothilone D (also known as Desoxyepothilone F and dEpoF), 26-fluoroepothilone); FR-182877 (Fujisawa, also known as WS-9885B), BSF-223651 (BASF, also known as ILX-651 and LU-223651); AC-7739 (Ajinomoto, also known as AVE-8063A and CS-39.HCl); AC-7700 (Ajinomoto, also known as AVE-8062, AVE-8062A, CS-39-L-Ser.HCl, and RPR-258062A); Fijianolide B; Laulimalide; Caribaeoside; Caribaeolin; Taccalonolide; Eleutherobin; Sarcodictyin; Laulimalide; Dictyostatin-1; Jatrophane esters; and analogs and derivatives thereof.

As used herein, a “microtubulin inhibitor” means an anti-cancer agent which acts by inhibiting tubulin polymerization or microtubule assembly. Examples of microtubulin inhibitors include without limitation the following marketed drugs and drugs in development: Erbulozole (also known as R-55104); Dolastatin 10 (also known as DLS-10 and NSC-376128); Mivobulin isethionate (also known as CI-980); Vincristine; NSC-639829; ABT-751 (Abbot, also known as E-7010); Altorhyrtins (such as Altorhyrtin A and Altorhyrtin C); Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9); Cemadotin hydrochloride (also known as LU-103793 and NSC-D-669356); Auristatin PE (also known as NSC-654663); Soblidotin (also known as TZT-1027), LS-4559-P (Pharmacia, also known as LS-4577); LS-4578 (Pharmacia, also known as LS-477-P); LS-4477 (Pharmacia), LS-4559 (Pharmacia); RPR-112378 (Aventis); Vincristine sulfate; DZ-3358 (Daiichi); GS-164 (Takeda); GS-198 (Takeda); KAR-2 (Hungarian Academy of Sciences); SAH-49960 (Lilly/Novartis); SDZ-268970 (Lilly/Novartis); AM-97 (Armad/Kyowa Hakko); AM-132 (Armad); AM-138 (Armad/Kyowa Hakko); IDN-5005 (Indena); Cryptophycin 52 (also known as LY-355703); Vitilevuamide; Tubulysin A; Canadensol; Centaureidin (also known as NSC-106969); T-138067 (Tularik, also known as T-67, TL-138067 and TI-138067); COBRA-1 (Parker Hughes Institute, also known as DDE-261 and WHI-261); H10 (Kansas State University); H16 (Kansas State University); Oncocidin AI (also known as BTO-956 and DIME); DDE-313 (Parker Hughes Institute); SPA-2 (Parker Hughes Institute); SPA-1 (Parker Hughes Institute, also known as SPIKET-P); 3-IAABU (Cytoskeleton/Mt. Sinai School of Medicine, also known as MF-569); Narcosine (also known as NSC-5366); Nascapine, D-24851 (Asta Medica), A-105972 (Abbott); Hemiasterlin; 3-BAABU (Cytoskeleton/Mt. Sinai School of Medicine, also known as MF-191); TMPN (Arizona State University); Vanadocene acetylacetonate; T-138026 (Tularik); Monsatrol; Inanocine (also known as NSC-698666); 3-IAABE (Cytoskeleton/Mt. Sinai School of Medicine); A-204197 (Abbott); T-607 (Tularik, also known as T-900607); RPR-115781 (Aventis); Eleutherobins (such as Desmethyleleutherobin, Desaetyleleutherobin, Isoeleutherobin A, and Z-Eleutherobin); Halichondrin B; D-64131 (Asta Medica); D-68144 (Asta Medica); Diazonamide A; A-293620 (Abbott); NPI-2350 (Nereus); TUB-245 (Aventis); A-259754 (Abbott); Diozostatin; (−)-Phenylahistin (also known as NSCL-96F037); D-68838 (Asta Medica); D-68836 (Asta Medica); Myoseverin B; D-43411 (Zentaris, also known as D-81862); A-289099 (Abbott); A-318315 (Abbott); HTI-286 (also known as SPA-110, trifluoroacetate salt) (Wyeth); D-82317 (Zentaris); D-82318 (Zentaris); SC-12983 (NCI); Resverastatin phosphate sodium; BPR-0Y-007 (National Health Research Institutes); SSR-250411 (Sanofi); Combretastatin A4; and analogs and derivatives thereof.

TAXOL®, also referred to as “paclitaxel”, is a well-known anti-cancer drug which acts by enhancing and stabilizing microtubule formation. Many analogs of TAXOL® are known, including TAXOTERE®. TAXOTERE® is also referred to as “docetaxel”. The structures of other TAXOL® analogs are shown in below (and in U.S. application Ser. No. 11/157,213 the entire contents of which are incorporated herein by reference):

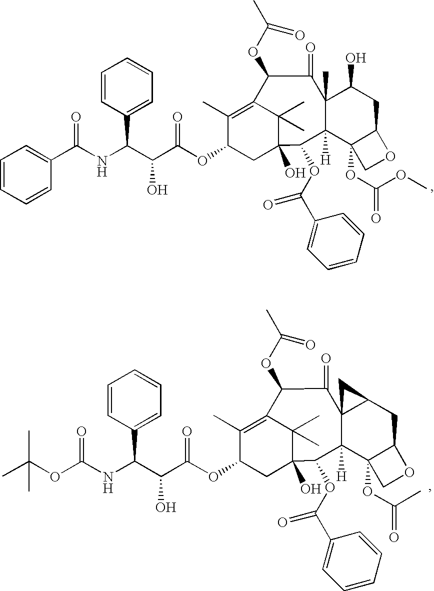

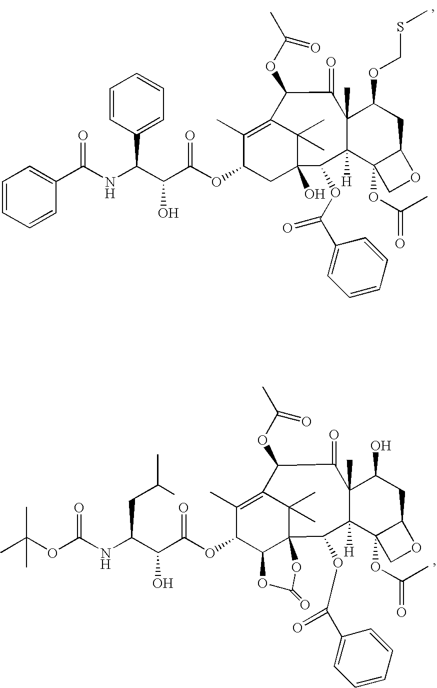

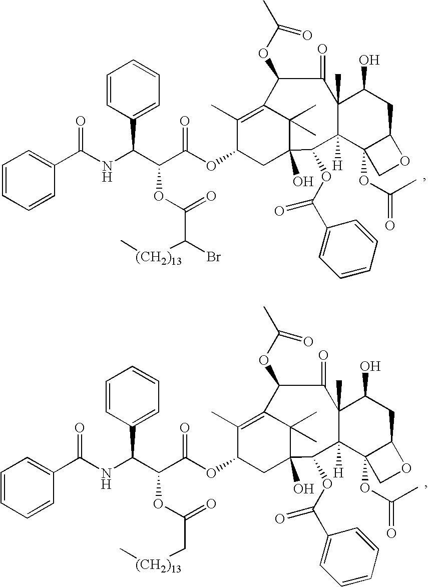

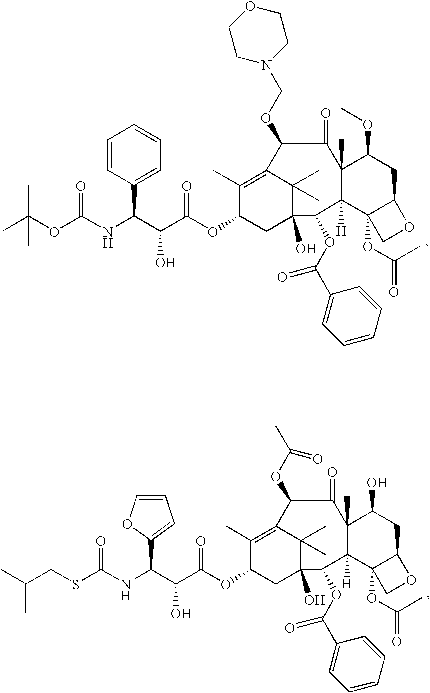

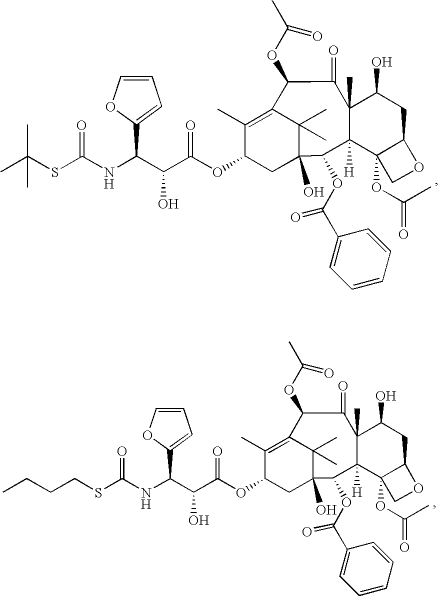

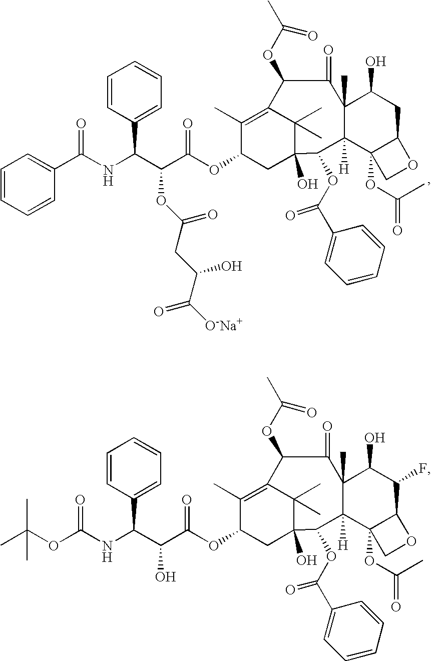

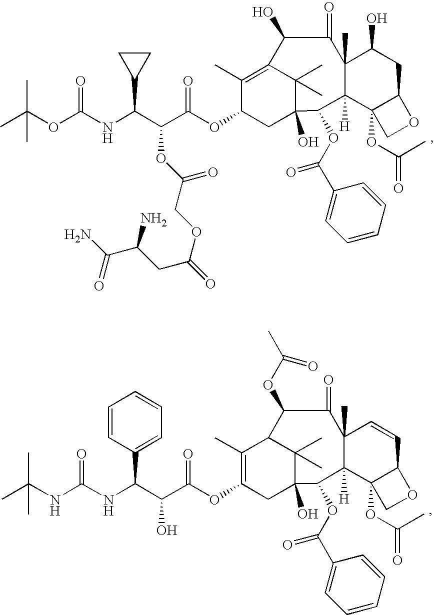

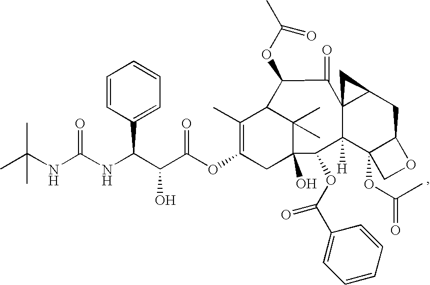

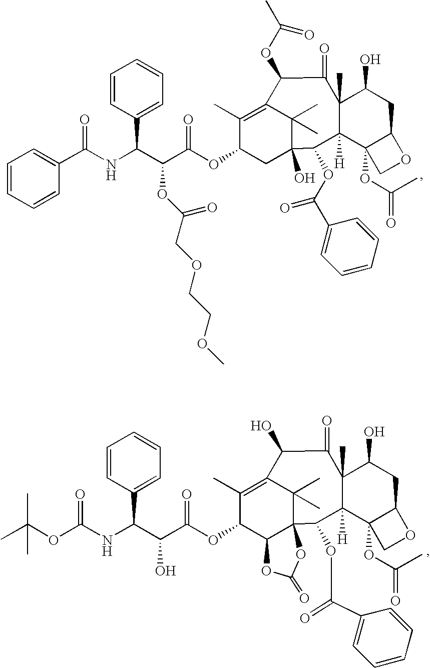

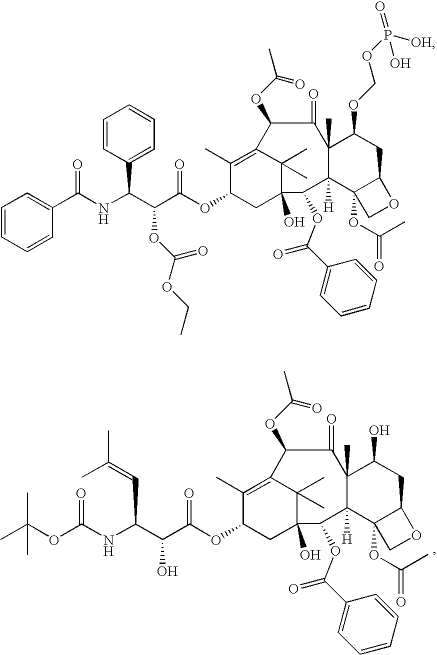

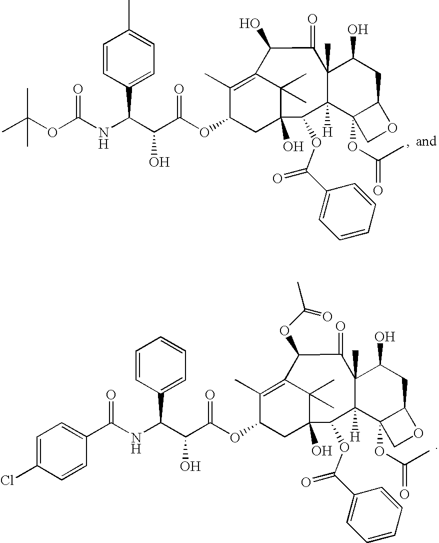

These compounds have the basic taxane skeleton as a common structure feature and have also been shown to have the ability to arrest cells in the G2-M phases due to stabilization of microtubules. Thus, a wide variety of substituents can decorate the taxane skeleton without adversely affecting biological activity. It is also apparent that zero, one or both of the cyclohexane rings of a TAXOL® analog can have a double bond at the indicated positions. For clarity purposes, the basic taxane skeleton is shown below in Structural Formula (X):

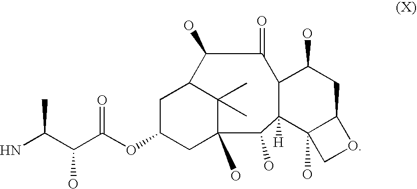

Double bonds have been omitted from the cyclohexane rings in the taxane skeleton represented by Structural Formula (X). The basic taxane skeleton can include zero or one double bond in one or both cyclohexane rings, as indicated in Structural Formulas (XI) and (XII) below. A number of atoms have also been omitted from Structural Formula (X) to indicate sites in which structural variation commonly occurs among TAXOL® analogs. For example, substitution on the taxane skeleton with simply an oxygen atom indicates that hydroxyl, acyl, alkoxy or another oxygen-bearing substituent is commonly found at the site. These and other substitutions on the taxane skeleton can be made without losing the ability to enhance and stabilize microtubule formation. Thus, the term “taxol analog” is defined herein to mean a compound which has the basic taxol skeleton and which promotes microtubule formation. TAXOL® analogs may be formulated as a nanoparticle colloidal composition to improve the infusion time and to eliminate the need to deliver the drug with Cremophor which causes hypersensitivity reactions in some patients. An example of a TAXOL® analog formulated as a nanoparticle colloidal composition is ABI-007 which is a nanoparticle colloidal composition of protein-stabilized paclitaxel that is reconstituted in saline.

Typically, the TAXOL® analogs used herein are represented by Structural Formula (XI) or (XII):

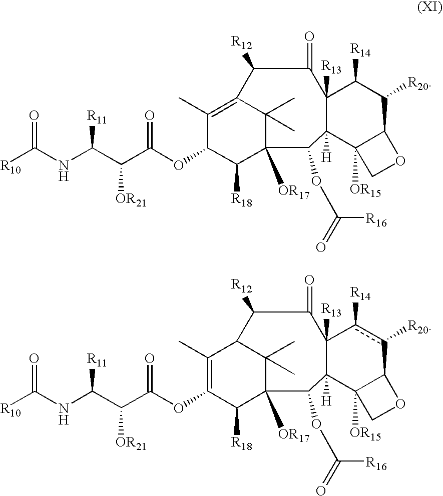

### R₁₀ is a lower alkyl group, a substituted lower alkyl group, a phenyl group, a substituted phenyl group, —SR₁₉, —NHR₁₉ or —OR₁₉.

R₁₁ is a lower alkyl group, a substituted lower alkyl group, an aryl group or a substituted aryl group.

R₁₂ is —H, —OH, lower alkyl, substituted lower alkyl, lower alkoxy, substituted lower alkoxy, —O—C(O)-(lower alkyl), —O—C(O)-(substituted lower alkyl), —O—CH₂—O-(lower alkyl)-S—CH₂—O-(lower alkyl).

R₁₃ is —H, —CH₃, or, taken together with R₁₄, —CH₂—.

R₁₄ is —H, —OH, lower alkoxy, —O—C(O)-(lower alkyl), substituted lower alkoxy, —O—C(O)-(substituted lower alkyl), —O—CH₂—O—P(O)(OH)₂, —O—CH₂—O-(lower alkyl), —O—CH₂—S-(lower alkyl) or, taken together with R₂₀, a double bond.

R₁₅—H, lower acyl, lower alkyl, substituted lower alkyl, alkoxymethyl, alkthiomethyl, —OC(O)—O(lower alkyl), —OC(O)—O(substituted lower alkyl), —OC(O)—NH(lower alkyl) or —OC(O)—NH(substituted lower alkyl).

R₁₆ is phenyl or substituted phenyl.

R₁₇ is —H, lower acyl, substituted lower acyl, lower alkyl, substituted, lower alkyl, (lower alkoxy)methyl or (lower alkyl)thiomethyl.

R₁₈—H, —CH₃ or, taken together with R₁₇ and the carbon atoms to which R₁₇ and R₁₈ are bonded, a five or six membered a non-aromatic heterocyclic ring.

R₁₉ is a lower alkyl group, a substituted lower alkyl group, a phenyl group, a substituted phenyl group.

R₂₀ is —H or a halogen.

R₂₁ is —H, lower alkyl, substituted lower alkyl, lower acyl or substituted lower acyl.

Preferably, the variables in Structural Formulas (XI) and (XII) are defined as follows: R₁₀ is phenyl, tert-butoxy, —S—CH₂—CH—(CH₃)₂, —S—CH(CH₃)₃, —S—(CH₂)₃CH₃, —O—CH(CH₃)₃, —NH—CH(CH₃)₃, —CH═C(CH₃)₂ or para-chlorophenyl; R₁₁ is phenyl, (CH₃)₂CHCH₂—, -2-furanyl, cyclopropyl or para-toluoyl; R₁₂ is —H, —OH, CH₃CO— or —(CH₂)₂—N-morpholino; R₁₃ is methyl, or, R₁₃ and R₁₄, taken together, are —CH₂—;

R₁₄ is —H, —CH₂SCH₃ or —CH₂—O—P(O)(OH)₂; R₁₅ is CH₃CO—;

R₁₆ is phenyl; R₁₇—H, or, R₁₇ and R₁₈, taken together, are —O—CO—O—;

R₁₈ is —H; R₂₀ is —H or —F; and R₂, is —H, —C(O)—CHBr—(CH₂)₁₃—CH₃ or —C(O)—(CH₂)₁₄—CH₃; —C(O)—CH₂—CH(OH)—COOH, —C(O)—CH₂—O—C(O)—CH₂CH(NH₂)—CONH₂, —C(O)—CH₂—O—CH₂CH₂OCH₃ or —C(O)—O—C(O)—CH₂CH₃.

A TAXOL® analog can also be bonded to or be pendent from a pharmaceutically acceptable polymer, such as a polyacrylamide. One example of a polymer of this type is shown in US Application Publication No. 2006/0135595. The term “taxol analog”, as it is used herein, includes such polymers.

In some embodiments, TAXOL® analogs have a taxane skeleton represented by Structural Formula IX, wherein Z is O, S, or NR. TAXOL® analogs that have the taxane skeleton shown in Structural Formula IX can have various substituents attached to the taxane skeleton and can have a double bond in zero, one or both of the cyclohexane rings as shown, for example in structure above.

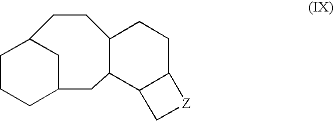

Various TAXOL® analogs and TAXOL® formulations are described in Hennenfent et al. (2006) Annals of Oncology 17:735-749; Gradishar (2006) Expert Opin. Pharmacother. 7(8):1041-53; Attard et al. (2006) Pathol Biol 54(2):72-84; Straubinger et al. (2005) Methods Enzymol. 391:97-117; Ten Tije et al. (2003) Clin Pharmacokinet. 42(7):665-85; and Nuijen et al. (2001) Invest New Drugs. 19(2):143-53, the entire teachings of which are incorporated herein by reference.

The compositions of the present invention can be administered by, for example, oral, topical, rectal, vaginal, nasal, pulmonary or parenteral (injection, infusion) administration.

In addition to the formulations described above, a formulation can optionally include, preserving agents, solubilizing agents, chemical buffers, surfactants, emulsifiers, colorants, odorants and sweeteners.

A “subject” is a mammal, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

As noted above, one embodiment of the present invention is directed to treating subjects with cancer. “Treating a subject with cancer” includes achieving, partially or substantially, one or more of the following results: arresting the growth or spread of a cancer, reducing the extent of a cancer (e.g., reducing size of a tumor or reducing the number of affected sites), inhibiting the growth rate of a cancer, and ameliorating or improving a clinical symptom or indicator associated with a cancer. “Treating a subject with cancer” also includes partially or totally inhibiting, delaying or preventing the progression of cancer including cancer metastasis; partially or totally inhibiting, delaying or preventing the recurrence of cancer including cancer metastasis (in a subject who has been treated for cancer); or partially or totally preventing the onset or development of cancer (chemoprevention). Partially or totally inhibiting, delaying or preventing the recurrence of means inhibiting, delaying or preventing the recurrence of the cancer, after the original tumor has been removed, for example, by surgery. A subject who has been “treated for cancer”, is a subject in which, for example, the primary tumor has been, for example, removed surgically or has gone into remission following treatment by, for example, chemotherapy.

The term “effective amount” is the quantity of compound in which a beneficial clinical outcome is achieved when the compound is administered to a subject with a cancer. A “beneficial clinical outcome” includes prevention, inhibition or a delay in the recurrence of cancer, a reduction in tumor mass, a reduction in metastasis, a reduction in the severity of the symptoms associated with the cancer and/or an increase in the longevity of the subject compared with the absence of the treatment. The precise amount of bis(thiohydrazide amide) administered to a subject will depend on the type and severity of the disease or condition and on the characteristics of the subject, such as general health, age, sex, body weight and tolerance to drugs. It will also depend on the degree, severity and type of cancer. The skilled artisan will be able to determine appropriate dosages depending on these and other factors.

Effective amounts of the disclosed bis(thiohydrazide amides) typically range between about 1 mg/mm² per day and about 10 grams/mm² per day, and preferably between 10 mg/mm² per day and about 5 grams/mm². When co-administered with an immunotherapy or another anti-cancer agent, an “effective amount” of the immunotherapy or anti-cancer agent will depend on the type of drug used. Suitable dosages are known for approved anti-cancer agents and can be adjusted by the skilled artisan according to the condition of the subject, the type of cancer being treated and the amount of bis(thio-hydrazide amide) disalt being used.

Examples of specific dosage regimens for the disclosed compounds used in combination with taxanes are provided below.

One dosage regimen includes the step of co-administering to the subject over three to five weeks, a taxane in an amount of between about 243 μmol/m2 to 315 μmol/m2 (e.g., equivalent to paclitaxel in about 210-270 mg/m2); and a bis(thiohydrazide amide) (e.g., as represented by Structural Formula I) in an amount between about 1473 μmol/m2 and about 1722 μmol/m2 (e.g., Compound (1) in about 590-690 mg/m2).

In another dosage regimen the taxane and the bis(thio-hydrazide) amide can each be administered in three equal weekly doses for three weeks of a four week period. In preferred embodiments, the four week administration period can be repeated until the cancer is in remission. The taxane can be any taxane defined herein. In a specific embodiment, the taxane is paclitaxel intravenously administered in a weekly dose of about 94 μmol/m2 (80 mg/m2). Typically, the bis(thiohydrazide amide) can be intravenously administered in a weekly dose of between about 500 μmol/m2 and about 562 μmol/m2, or more typically in a weekly dose of about 532 μmol/m2. (e.g., Compound (1) in about 590-690 mg/m2).

Another dosage regimen includes intravenously administering to the subject in a four week period, three equal weekly doses of paclitaxel in an amount of about 94 μmol/m2; and compound (1) or a pharmaceutically acceptable salt or solvate thereof in an amount of about 532 μmol/m2.

In another dosage regimen, the subject can be intravenously administered between about 220 μmol/m2 and about 1310 μmol/m2 (e.g., Compound (1) in about 88-525 mg/m2) of the bis(thiohydrazide amide) once every 3 weeks, generally between about 220 μmol/m2 and about 1093 μmol/m2 (e.g., Compound (1) in about 88-438 mg/m2) once every 3 weeks, typically between about 624 μmol/m2 and about 1124 μmol/m2 m2 (e.g., Compound (1) in about 250-450 mg/m2), more typically between about 811 μmol/m2 and about 936 μmol/m2 m2 (e.g., Compound (1) in about 325-375 mg/m2), or in particular embodiments, about 874 μmol/m2 ((e.g., Compound (1) in about 350 mg/m2). In particular embodiments, the subject can be intravenously administered between about 582 μmol/m2 and about 664 μmol/m2 (e.g., Compound (1) in about 233-266 mg/m2) of the bis(thiohydrazide amide) once every 3 weeks. In certain embodiments, the bis(thiohydrazide amide) is in an amount of about 664 μmol/m2 (e.g., Compound (1) in about 266 mg/m2).

In another dosage regimen, the subject can be intravenously administered between about 200 μmol/m2 to about 263 μmol/m2 of the taxane as paclitaxel once every 3 weeks (e.g., paclitaxel in about 175-225 mg/m2). In some embodiments, the subject can be intravenously administered between about 200 μmol/m2 to about 234 μmol/m2 of the taxane as paclitaxel once every 3 weeks (e.g., paclitaxel in about 175-200 mg/m2). In certain embodiments, the paclitaxel is administered in an amount of about 234 μmol/m2 (200 mg/m2). In certain embodiments, the paclitaxel is administered in an amount of about 205 μmol/m2 (175 mg/m2).

In one embodiment, the taxane, e.g., paclitaxel, and the bis(thiohydrazide amide), e.g., Compound (1), can be administered together in a single pharmaceutical composition.

In one embodiment, the method of the present invention includes treating a subject once every three weeks, independently or together a taxane in an amount of about 205 μmol/m2 (e.g., paclitaxel in about 175 mg/m2); and a bis(thiohydrazide amide) represented by Structural Formula I or a pharmaceutically acceptable salt or solvate thereof in an amount between about 220 μmol/m2 and about 1310 μmol/m2 (e.g., Compound (1) in about 88-525 mg/m2). Typically, the taxane is paclitaxel intravenously administered in an amount of about 205 μmol/m2. The bis(thiohydrazide amide) can typically be intravenously administered between about 220 μmol/m2 and about 1093 μmol/m2 (e.g., Compound (1) in about 88-438 mg/m2), more typically between about 749 μmol/m2 and about 999 μmol/m2 (e.g., compound (1) in about 300-400 mg/m2), in some embodiments between about 811 μmol/m2 and about 936 μmol/m2 (e.g., Compound (1) in about 325-375 mg/m2). In certain embodiments, the bis(thiohydrazide amide) can be Compound (1) intravenously administered between about 874 μmol/m2 (about 350 mg/m2).

In a particular embodiment, the methods of the present invention involve intravenously administering to the subject in a single dose per three week period: paclitaxel in an amount of about 205 μmol/m2 (175 mg/m2); and Compound (1) or a pharmaceutically acceptable salt or solvate thereof in an amount of about 874 μmol/m2 (350 mg/m2).

Particular formulations, dosages and modes of administration are as described in US Publication No. 20060135595 and PCT/US2006/014531 filed 13 Apr. 2006, titled Combination Cancer Therapy With Bis[Thiohydrazide] Amide Compounds the entire contents of each of which are incorporated herein by reference).

The bis(thio-hydrazide amide) disclosed herein can be prepared by the methods described in U.S. Publication Nos. 20060135595, 2003/0045518 and 2003/0119914, U.S. application Ser. No. 11/432,307, filed 11 May 2006, titled Synthesis Of Bis(Thio-Hydrazide Amide) Salts, U.S. Provisional Patent No. 60/708,977 filed 16 Aug. 2005, titled Bis(Thio-Hydrazide Amide) Formulation and also according to methods described in U.S. Publication No. 2004/0225016 A1, entitled TREATMENT FOR CANCERS. The entire teachings of these applications are incorporated herein by reference.

While this invention has been particularly shown and described with references to example embodiments thereof, it will be understood by those skilled in the art that various changes in form and details may be made therein without departing from the scope of the invention encompassed by the appended claims.

1. A composition comprising a compound represented by the following Structural Formula: 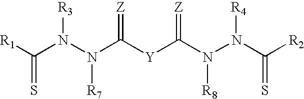
or a pharmaceutically acceptable salt or solvate thereof, wherein:
Y is a covalent bond or an optionally substituted straight chained hydrocarbyl group, or, Y, taken together with both >C=Z groups to which it is bonded, is an optionally substituted aromatic group;
R₁-R₄ are independently —H, an optionally substituted aliphatic group, an optionally substituted aryl group, or R₁ and R₃ taken together with the carbon and nitrogen atoms to which they are bonded, and/or R₂ and R₄ taken together with the carbon and nitrogen atoms to which they are bonded, form a non-aromatic heterocyclic ring optionally fused to an aromatic ring;
R₇-R₈ are independently —H, an optionally substituted aliphatic group, or an optionally substituted aryl group;
Z is O or S;
wherein the compound is substantially or completely encased in a polymeric shell.

2-29. (canceled)

30. The composition of claim 1, wherein the compound is represented by the following Structural Formula: 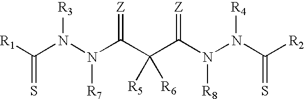
or a pharmaceutically acceptable salt or solvate thereof, wherein:
R₇-R₈ are both —H, and:
R₁ and R₂ are both phenyl, R₃ and R₄ are both methyl, and R₅ and R₆ are both —H;
R₁ and R₂ are both phenyl, R₃ and R₄ are both ethyl, and R₅ and R₆ are both —H;
R₁ and R₂ are both 4-cyanophenyl, R₃ and R₄ are both methyl, R₅ is methyl, and R₆ is —H;
R₁ and R₂ are both 4-methoxyphenyl, R₃ and R₄ are both methyl, and R₅ and R₆ are both —H;
R₁ and R₂ are both phenyl, R₃ and R₄ are both methyl, R₅ is methyl, and R₆ is —H;
R₁ and R₂ are both phenyl, R₃ and R₄ are both ethyl, R₅ is methyl, and R₆ is —H;
R₁ and R₂ are both 4-cyanophenyl, R₃ and R₄ are both methyl, and R₅ and R₆ are both —H;
R₁ and R₂ are both 2,5-dimethoxyphenyl, R₃ and R₄ are both methyl, and R₅ and R₆ are both —H;
R₁ and R₂ are both 2,5-dimethoxyphenyl, R₃ and R₄ are both methyl, R₅ is methyl, and R₆ is —H;
R₁ and R₂ are both 3-cyanophenyl, R₃ and R₄ are both methyl, and R₅ and R₆ are both —H;
R₁ and R₂ are both 3-fluorophenyl, R₃ and R₄ are both methyl, and R₅ and R₆ are both —H;
R₁ and R₂ are both 4-chlorophenyl, R₃ and R₄ are both methyl, R₅ is methyl, and R₆ is —H;
R₁ and R₂ are both 2-dimethoxyphenyl, R₃ and R₄ are both methyl, and R₅ and R₆ are both —H;
R₁ and R₂ are both 3-methoxyphenyl, R₃ and R₄ are both methyl, and R₅ and R₆ are both —H;
R₁ and R₂ are both 2,3-dimethoxyphenyl, R₃ and R₄ are both methyl, and R₅ and R₆ are both —H;
R₁ and R₂ are both 2,3-dimethoxyphenyl, R₃ and R₄ are both methyl, R₅ is methyl, and R₆ is —H;
R₁ and R₂ are both 2,5-difluorophenyl, R₃ and R₄ are both methyl, and R₅ and R₆ are both —H;
R₁ and R₂ are both 2,5-difluorophenyl, R₃ and R₄ are both methyl, R₅ is methyl, and R₆ is —H;
R₁ and R₂ are both 2,5-dichlorophenyl, R₃ and R₄ are both methyl, and R₅ and R₆ are both —H;
R₁ and R₂ are both 2,5-dimethylphenyl, R₃ and R₄ are both methyl, and R₅ and R₆ are both —H;
R₁ and R₂ are both 2,5-dimethoxyphenyl, R₃ and R₄ are both methyl, and R₅ and R₆ are both —H;
R₁ and R₂ are both phenyl, R₃ and R₄ are both methyl, and R₅ and R₆ are both —H;
R₁ and R₂ are both 2,5-dimethoxyphenyl, R₃ and R₄ are both methyl, R₅ is methyl, and R₆ is —H;
R₁ and R₂ are both cyclopropyl, R₃ and R₄ are both methyl, and R₅ and R₆ are both —H;
R₁ and R₂ are both cyclopropyl, R₃ and R₄ are both ethyl, and R₅ and R₆ are both —H;
R₁ and R₂ are both cyclopropyl, R₃ and R₄ are both methyl, R₅ is methyl, and R₆ is —H;
R₁ and R₂ are both 1-methylcyclopropyl, R₃ and R₄ are both methyl, and R₅ and R₆ are both —H;
R₁ and R₂ are both 1-methylcyclopropyl, R₃ and R₄ are both methyl, R₅ is methyl and R₆ is —H;
R₁ and R₂ are both 1-methylcyclopropyl, R₃ and R₄ are both methyl, R₅ is ethyl, and R₆ is —H;
R₁ and R₂ are both 1-methylcyclopropyl, R₃ and R₄ are both methyl, R₅ is n-propyl, and R₆ is —H;
R₁ and R₂ are both 1-methylcyclopropyl, R₃ and R₄ are both methyl, and R₅ and R₆ are both methyl;
R₁ and R₂ are both 1-methylcyclopropyl, R₃ and R₄ are both ethyl, and R₅ and R₆ are both —H;
R₁ and R₂ are both 1-methylcyclopropyl, R₃ is methyl, R₄ is ethyl, and R₅ and R₆ are both —H;
R₁ and R₂ are both 2-methylcyclopropyl, R₃ and R₄ are both methyl, and R₅ and R₆ are both —H;
R₁ and R₂ are both 2-phenylcyclopropyl, R₃ and R₄ are both methyl, and R₅ and R₆ are both —H;
R₁ and R₂ are both 1-phenylcyclopropyl, R₃ and R₄ are both methyl, and R₅ and R₆ are both —H;
R₁ and R₂ are both cyclobutyl, R₃ and R₄ are both methyl, and R₅ and R₆ are both —H;
R₁ and R₂ are both cyclopentyl, R₃ and R₄ are both methyl, and R₅ and R₆ are both —H;
R₁ and R₂ are both cyclohexyl, R₃ and R₄ are both methyl, and R₅ and R₆ are both —H;
R₁ and R₂ are both cyclohexyl, R₃ and R₄ are both phenyl, and R₅ and R₆ are both —H;
R₁ and R₂ are both methyl, R₃ and R₄ are both methyl, and R₅ and R₆ are both —H;
R₁ and R₂ are both methyl, R₃ and R₄ are both t-butyl, and R₅ and R₆ are both —H;
R₁ and R₂ are both methyl, R₃ and R₄ are both phenyl, and R₅ and R₆ are both —H;
R₁ and R₂ are both t-butyl, R₃ and R₄ are both methyl, and R₅ and R₆ are both —H;
R₁ and R₂ are ethyl, R₃ and R₄ are both methyl, and R₅ and R₆ are both —H; or
R₁ and R₂ are both n-propyl, R₃ and R₄ are both methyl, and R₅ and R₆ are both —H.

31. The composition of claim 1, wherein the compound is represented by the following Structural Formula: 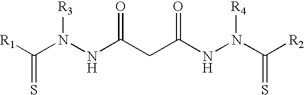
or a pharmaceutically acceptable salt thereof.

32. The composition of claim 1, wherein the compound is represented by one of the following Structural Formulas: 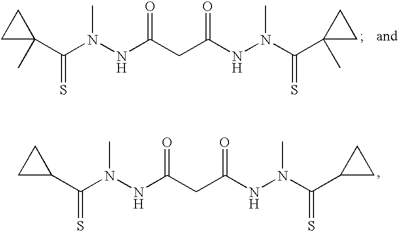
or a pharmaceutically acceptable salt thereof.

33. The composition of claim 1, wherein the compound is represented by the following Structural Formula: 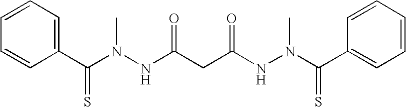
or a pharmaceutically acceptable salt thereof.

34. The composition of claim 33, wherein the compound is a disodium or a dipotassium salt.

35. The composition of claim 1, further comprising a microtubulin stabilizer selected from the group consisting of taxol, taxol analogues, Discodermolide (also known as NVP-XX-A-296); Epothilones (such as Epothilone A, Epothilone B, Epothilone C (also known as desoxyepothilone A or dEpoA); Epothilone D (also referred to as KOS-862, dEpoB, and desoxyepothilone B); Epothilone E; Epothilone F; Epothilone B N-oxide; Epothilone A N-oxide; 16-aza-epothilone B; 21-aminoepothilone B (also known as BMS-310705); 21-hydroxyepothilone D (also known as Desoxyepothilone F and dEpoF), 26-fluoroepothilone); FR-182877 (Fujisawa, also known as WS-9885B), BSF-223651 (BASF, also known as ILX-651 and LU-223651); AC-7739 (Ajinomoto, also known as AVE-8063A and CS-39.HCl); AC-7700 (Ajinomoto, also known as AVE-8062, AVE-8062A, CS-39-L-Ser.HCl, and RPR-258062A); Fijianolide B; Laulimalide; Caribaeoside; Caribaeolin; Taccalonolide; Eleutherobin; Sarcodictyin; Laulimalide; Dictyostatin-1; Jatrophane esters; and analogs and derivatives thereof, wherein the microtubulin stabilizer is substantially or completely encased in the polymeric shell

36. The composition of claim 35, wherein the microtubulin stabilizer is taxol or a taxol analog.

37-40. (canceled)

41. The composition of claim 36, wherein the taxol analog is taxotere.

42. A composition comprising a compound represented by the following Structural Formula: 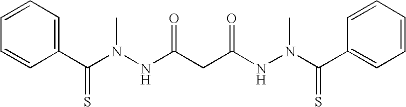
or a pharmaceutically acceptable salt thereof,
wherein the compound is substantially or completely encased in a biocompatible polymeric shell, wherein the biocompatible polymeric shell is albumin substantially crosslinked by disulfide bonds.

43-50. (canceled)

51. A composition comprising a compound represented by the following Structural Formula: 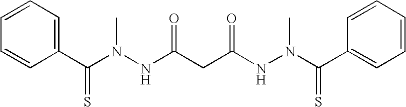
or a pharmaceutically acceptable salt thereof, and taxol or taxotere,
wherein the compound and taxol or taxotere are substantially or completely encased in a biocompatible polymeric shell, wherein the biocompatible polymeric shell is albumin substantially crosslinked by disulfide bonds.

52. The composition of claim 1, wherein the average diameter of the polymeric shell is less than about 100 microns.

53. A drug delivery device comprising particles of a compound represented by the following Structural Formula: 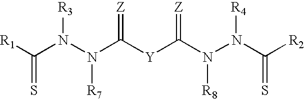
or a pharmaceutically acceptable salt or solvate thereof, wherein:
Y is a covalent bond or an optionally substituted straight chained hydrocarbyl group, or, Y, taken together with both >C=Z groups to which it is bonded, is an optionally substituted aromatic group;
R₁-R₄ are independently —H, an optionally substituted aliphatic group, an optionally substituted aryl group, or R₁ and R₃ taken together with the carbon and nitrogen atoms to which they are bonded, and/or R₂ and R₄ taken together with the carbon and nitrogen atoms to which they are bonded, form a non-aromatic heterocyclic ring optionally fused to an aromatic ring;
R₇-R₈ are independently —H, an optionally substituted aliphatic group, or an optionally substituted aryl group;
Z is O or S,
coated with a protein, wherein the protein has free protein associated therewith;
wherein a portion of said compound is contained within said protein coating and a portion of said compound is associated with said free protein.

54-74. (canceled)

75. The drug delivery device of claim 53, wherein the compound is represented by the following Structural Formula: 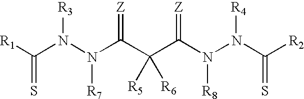
or a pharmaceutically acceptable salt or solvate thereof, wherein:
R₇-R₈ are both —H, and:
R₁ and R₂ are both phenyl, R₃ and R₄ are both methyl, and R₅ and R₆ are both —H;
R₁ and R₂ are both phenyl, R₃ and R₄ are both ethyl, and R₅ and R₆ are both —H;
R₁ and R₂ are both 4-cyanophenyl, R₃ and R₄ are both methyl, R₅ is methyl, and R₆ is —H;
R₁ and R₂ are both 4-methoxyphenyl, R₃ and R₄ are both methyl, and R₅ and R₆ are both —H;
R₁ and R₂ are both phenyl, R₃ and R₄ are both methyl, R₅ is methyl, and R₆ is —H;
R₁ and R₂ are both phenyl, R₃ and R₄ are both ethyl, R₅ is methyl, and R₆ is —H;
R₁ and R₂ are both 4-cyanophenyl, R₃ and R₄ are both methyl, and R₅ and R₆ are both —H;
R₁ and R₂ are both 2,5-dimethoxyphenyl, R₃ and R₄ are both methyl, and R₅ and R₆ are both —H;
R₁ and R₂ are both 2,5-dimethoxyphenyl, R₃ and R₄ are both methyl, R₅ is methyl, and R₆ is —H;
R₁ and R₂ are both 3-cyanophenyl, R₃ and R₄ are both methyl, and R₅ and R₆ are both —H;
R₁ and R₂ are both 3-fluorophenyl, R₃ and R₄ are both methyl, and R₅ and R₆ are both —H;
R₁ and R₂ are both 4-chlorophenyl, R₃ and R₄ are both methyl, R₅ is methyl, and R₆ is —H;
R₁ and R₂ are both 2-dimethoxyphenyl, R₃ and R₄ are both methyl, and R₅ and R₆ are both —H;
R₁ and R₂ are both 3-methoxyphenyl, R₃ and R₄ are both methyl, and R₅ and R₆ are both —H;
R₁ and R₂ are both 2,3-dimethoxyphenyl, R₃ and R₄ are both methyl, and R₅ and R₆ are both —H;
R₁ and R₂ are both 2,3-dimethoxyphenyl, R₃ and R₄ are both methyl, R₅ is methyl, and R₆ is —H;
R₁ and R₂ are both 2,5-difluorophenyl, R₃ and R₄ are both methyl, and R₅ and R₆ are both —H;
R₁ and R₂ are both 2,5-difluorophenyl, R₃ and R₄ are both methyl, R₅ is methyl, and R₆ is —H;
R₁ and R₂ are both 2,5-dichlorophenyl, R₃ and R₄ are both methyl, and R₅ and R₆ are both —H;
R₁ and R₂ are both 2,5-dimethylphenyl, R₃ and R₄ are both methyl, and R₅ and R₆ are both —H;
R₁ and R₂ are both 2,5-dimethoxyphenyl, R₃ and R₄ are both methyl, and R₅ and R₆ are both —H;
R₁ and R₂ are both phenyl, R₃ and R₄ are both methyl, and R₅ and R₆ are both —H;
R₁ and R₂ are both 2,5-dimethoxyphenyl, R₃ and R₄ are both methyl, R₅ is methyl, and R₆ is —H;
R₁ and R₂ are both cyclopropyl, R₃ and R₄ are both methyl, and R₅ and R₆ are both —H;
R₁ and R₂ are both cyclopropyl, R₃ and R₄ are both ethyl, and R₅ and R₆ are both —H;
R₁ and R₂ are both cyclopropyl, R₃ and R₄ are both methyl, R₅ is methyl, and R₆ is —H;
R₁ and R₂ are both 1-methylcyclopropyl, R₃ and R₄ are both methyl, and R₅ and R₆ are both —H;
R₁ and R₂ are both 1-methylcyclopropyl, R₃ and R₄ are both methyl, R₅ is methyl and R₆ is —H;
R₁ and R₂ are both 1-methylcyclopropyl, R₃ and R₄ are both methyl, R₅ is ethyl, and R₆ is —H;
R₁ and R₂ are both 1-methylcyclopropyl, R₃ and R₄ are both methyl, R₅ is n-propyl, and R₆ is —H;
R₁ and R₂ are both 1-methylcyclopropyl, R₃ and R₄ are both methyl, and R₅ and R₆ are both methyl;
R₁ and R₂ are both 1-methylcyclopropyl, R₃ and R₄ are both ethyl, and R₅ and R₆ are both —H;
R₁ and R₂ are both 1-methylcyclopropyl, R₃ is methyl, R₄ is ethyl, and R₅ and R₆ are both —H;
R₁ and R₂ are both 2-methylcyclopropyl, R₃ and R₄ are both methyl, and R₅ and R₆ are both —H;
R₁ and R₂ are both 2-phenylcyclopropyl, R₃ and R₄ are both methyl, and R₅ and R₆ are both —H;
R₁ and R₂ are both 1-phenylcyclopropyl, R₃ and R₄ are both methyl, and R₅ and R₆ are both —H;
R₁ and R₂ are both cyclobutyl, R₃ and R₄ are both methyl, and R₅ and R₆ are both —H;
R₁ and R₂ are both cyclopentyl, R₃ and R₄ are both methyl, and R₅ and R₆ are both —H;
R₁ and R₂ are both cyclohexyl, R₃ and R₄ are both methyl, and R₅ and R₆ are both —H;
R₁ and R₂ are both cyclohexyl, R₃ and R₄ are both phenyl, and R₅ and R₆ are both —H;
R₁ and R₂ are both methyl, R₃ and R₄ are both methyl, and R₅ and R₆ are both —H;
R₁ and R₂ are both methyl, R₃ and R₄ are both t-butyl, and R₅ and R₆ are both —H;
R₁ and R₂ are both methyl, R₃ and R₄ are both phenyl, and R₅ and R₆ are both —H;
R₁ and R₂ are both t-butyl, R₃ and R₄ are both methyl, and R₅ and R₆ are both —H;
R₁ and R₂ are ethyl, R₃ and R₄ are both methyl, and R₅ and R₆ are both —H; or
R₁ and R₂ are both n-propyl, R₃ and R₄ are both methyl, and R₅ and R₆ are both —H.

76. The drug delivery device of claim 53, wherein the compound is represented by the following Structural Formula: 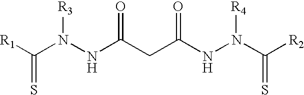
or a pharmaceutically acceptable salt thereof.

77. The drug delivery device of claim 53, wherein the compound is represented by one of the following Structural Formulas: 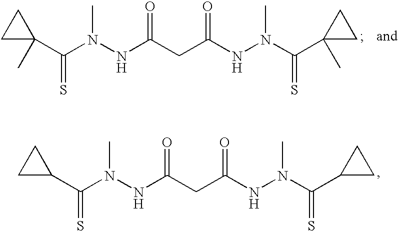
or a pharmaceutically acceptable salt thereof.

78. The drug delivery device of claim 53, wherein the compound is represented by the following Structural Formula: 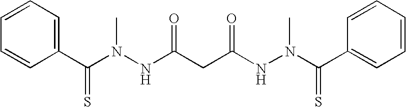
or a pharmaceutically acceptable salt thereof.

79. The drug delivery device of claim 78, wherein the compound is a disodium or a dipotassium salt.

80. The drug delivery device of claim 53, further comprising a microtubulin stabilizer selected from the group consisting of taxol; taxol analogues; Discodermolide (also known as NVP-XX-A-296); Epothilones (such as Epothilone A, Epothilone B, Epothilone C (also known as desoxyepothilone A or dEpoA); Epothilone D (also referred to as KOS-862, dEpoB, and desoxyepothilone B); Epothilone E; Epothilone F; Epothilone B N-oxide; Epothilone A N-oxide; 16-aza-epothilone B; 21-aminoepothilone B (also known as BMS-310705); 21-hydroxyepothilone D (also known as Desoxyepothilone F and dEpoF), 26-fluoroepothilone); FR-182877 (Fujisawa, also known as WS-9885B), BSF-223651 (BASF, also known as ILX-651 and LU-223651); AC-7739 (Ajinomoto, also known as AVE-8063A and CS-39.HCl); AC-7700 (Ajinomoto, also known as AVE-8062, AVE-8062A, CS-39-L-Ser.HCl, and RPR-258062A); Fijianolide B; Laulimalide; Caribaeoside; Caribaeolin; Taccalonolide; Eleutherobin; Sarcodictyin; Laulimalide; Dictyostatin-1; Jatrophane esters; and analogs and derivatives thereof, wherein, the microtubulin stabilizer is substantially or completely encased in the polymeric shell

81. The drug delivery device of claim 80, wherein the microtubulin stabilizer is taxol or a taxol analog.

82-85. (canceled)

86. The drug delivery device of claim 81, wherein the taxol analog is taxotere.

87. A drug delivery device comprising particles of a compound represented by the following Structural Formula: 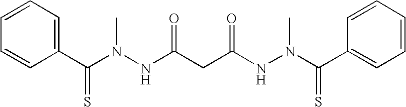
or a pharmaceutically acceptable salt thereof,
wherein the compound is coated with a protein, wherein said protein coating has free protein associated therewith; and
wherein a portion of said compound is contained within said protein coating and a portion of said compound is associated with said free protein and wherein said protein is albumin substantially crosslinked by disulfide bonds.

88-94. (canceled)

95. A drug delivery device comprising particles of a compound represented by the following Structural Formula: 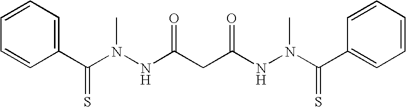
or a pharmaceutically acceptable salt thereof, and taxol or taxotere coated with a protein;
wherein said protein has free protein associate therewith; and
wherein a portion of the compound and a portion of the taxol or taxotere is contained within said protein coating and a portion the compound and a portion of the taxol or taxotere is associated with said free protein;
wherein the protein is albumin substantially crosslinked by disulfide bonds.

96. (canceled)

97. A composition prepared by subjecting an organic phase comprising a compound represented by the following Structural Formula: 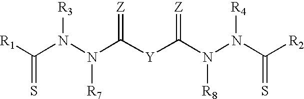
or a pharmaceutically acceptable salt or solvate thereof, wherein:
Y is a covalent bond or an optionally substituted straight chained hydrocarbyl group, or, Y, taken together with both >C=Z groups to which it is bonded, is an optionally substituted aromatic group;
R¹-R₄ are independently —H, an optionally substituted aliphatic group, an optionally substituted aryl group, or R₁ and R₃ taken together with the carbon and nitrogen atoms to which they are bonded, and/or R₂ and R₄ taken together with the carbon and nitrogen atoms to which they are bonded, form a non-aromatic heterocyclic ring optionally fused to an aromatic ring;
R₇-R₈ are independently —H, an optionally substituted aliphatic group, or an optionally substituted aryl group;
Z is O or S;
and an aqueous medium comprising a polymer, to sonication conditions for a time sufficient to promote crosslinking of the polymer by disulfide bonds to produce a polymeric shell encasing the compound substantially or completely.

98-128. (canceled)

129. The composition of claim 97, wherein the compound is represented by the following Structural Formula: 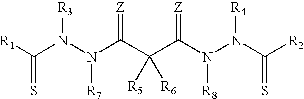
or a pharmaceutically acceptable salt or solvate thereof, wherein:
R₇-R₈ are both —H, and:
R₁ and R₂ are both phenyl, R₃ and R₄ are both methyl, and R₅ and R₆ are both —H;
R₁ and R₂ are both phenyl, R₃ and R₄ are both ethyl, and R₅ and R₆ are both —H;
R₁ and R₂ are both 4-cyanophenyl, R₃ and R₄ are both methyl, R₅ is methyl, and R₆ is —H;
R₁ and R₂ are both 4-methoxyphenyl, R₃ and R₄ are both methyl, and R₅ and R₆ are both —H;
R₁ and R₂ are both phenyl, R₃ and R₄ are both methyl, R₅ is methyl, and R₆ is —H;
R₁ and R₂ are both phenyl, R₃ and R₄ are both ethyl, R₅ is methyl, and R₆ is —H;
R₁ and R₂ are both 4-cyanophenyl, R₃ and R₄ are both methyl, and R₅ and R₆ are both —H;
R₁ and R₂ are both 2,5-dimethoxyphenyl, R₃ and R₄ are both methyl, and R₅ and R₆ are both —H;
R₁ and R₂ are both 2,5-dimethoxyphenyl, R₃ and R₄ are both methyl, R₅ is methyl, and R₆ is —H;
R₁ and R₂ are both 3-cyanophenyl, R₃ and R₄ are both methyl, and R₅ and R₆ are both —H;
R₁ and R₂ are both 3-fluorophenyl, R₃ and R₄ are both methyl, and R₅ and R₆ are both —H;
R₁ and R₂ are both 4-chlorophenyl, R₃ and R₄ are both methyl, R₅ is methyl, and R₆ is —H;
R₁ and R₂ are both 2-dimethoxyphenyl, R₃ and R₄ are both methyl, and R₅ and R₆ are both —H;
R₁ and R₂ are both 3-methoxyphenyl, R₃ and R₄ are both methyl, and R₅ and R₆ are both —H;
R₁ and R₂ are both 2,3-dimethoxyphenyl, R₃ and R₄ are both methyl, and R₅ and R₆ are both —H;
R₁ and R₂ are both 2,3-dimethoxyphenyl, R₃ and R₄ are both methyl, R₅ is methyl, and R₆ is —H;
R₁ and R₂ are both 2,5-difluorophenyl, R₃ and R₄ are both methyl, and R₅ and R₆ are both —H;
R₁ and R₂ are both 2,5-difluorophenyl, R₃ and R₄ are both methyl, R₅ is methyl, and R₆ is —H;
R₁ and R₂ are both 2,5-dichlorophenyl, R₃ and R₄ are both methyl, and R₅ and R₆ are both —H;
R₁ and R₂ are both 2,5-dimethylphenyl, R₃ and R₄ are both methyl, and R₅ and R₆ are both —H;
R₁ and R₂ are both 2,5-dimethoxyphenyl, R₃ and R₄ are both methyl, and R₅ and R₆ are both —H;
R₁ and R₂ are both phenyl, R₃ and R₄ are both methyl, and R₅ and R₆ are both —H;
R₁ and R₂ are both 2,5-dimethoxyphenyl, R₃ and R₄ are both methyl, R₅ is methyl, and R₆ is —H;
R₁ and R₂ are both cyclopropyl, R₃ and R₄ are both methyl, and R₅ and R₆ are both —H;
R₁ and R₂ are both cyclopropyl, R₃ and R₄ are both ethyl, and R₅ and R₆ are both —H;
R₁ and R₂ are both cyclopropyl, R₃ and R₄ are both methyl, R₅ is methyl, and R₆ is —H;
R₁ and R₂ are both 1-methylcyclopropyl, R₃ and R₄ are both methyl, and R₅ and R₆ are both —H;
R₁ and R₂ are both 1-methylcyclopropyl, R₃ and R₄ are both methyl, R₅ is methyl and R₆ is —H;
R₁ and R₂ are both 1-methylcyclopropyl, R₃ and R₄ are both methyl, R₅ is ethyl, and R₆ is —H;
R₁ and R₂ are both 1-methylcyclopropyl, R₃ and R₄ are both methyl, R₅ is n-propyl, and R₆ is —H;
R₁ and R₂ are both 1-methylcyclopropyl, R₃ and R₄ are both methyl, and R₅ and R₆ are both methyl;
R₁ and R₂ are both 1-methylcyclopropyl, R₃ and R₄ are both ethyl, and R₅ and R₆ are both —H;
R₁ and R₂ are both 1-methylcyclopropyl, R₃ is methyl, R₄ is ethyl, and R₅ and R₆ are both —H;
R₁ and R₂ are both 2-methylcyclopropyl, R₃ and R₄ are both methyl, and R₅ and R₆ are both —H;
R₁ and R₂ are both 2-phenylcyclopropyl, R₃ and R₄ are both methyl, and R₅ and R₆ are both —H;
R₁ and R₂ are both 1-phenylcyclopropyl, R₃ and R₄ are both methyl, and R₅ and R₆ are both —H;
R₁ and R₂ are both cyclobutyl, R₃ and R₄ are both methyl, and R₅ and R₆ are both —H;
R₁ and R₂ are both cyclopentyl, R₃ and R₄ are both methyl, and R₅ and R₆ are both —H;
R₁ and R₂ are both cyclohexyl, R₃ and R₄ are both methyl, and R₅ and R₆ are both —H;
R₁ and R₂ are both cyclohexyl, R₃ and R₄ are both phenyl, and R₅ and R₆ are both —H;
R₁ and R₂ are both methyl, R₃ and R₄ are both methyl, and R₅ and R₆ are both —H;
R₁ and R₂ are both methyl, R₃ and R₄ are both t-butyl, and R₅ and R₆ are both —H;
R₁ and R₂ are both methyl, R₃ and R₄ are both phenyl, and R₅ and R₆ are both —H;
R₁ and R₂ are both t-butyl, R₃ and R₄ are both methyl, and R₅ and R₆ are both —H;
R₁ and R₂ are ethyl, R₃ and R₄ are both methyl, and R₅ and R₆ are both —H; or
R₁ and R₂ are both n-propyl, R₃ and R₄ are both methyl, and R₅ and R₆ are both —H.

130. The composition of claim 97, wherein the compound is represented by the following Structural Formula: 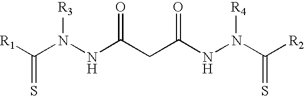
or a pharmaceutically acceptable salt thereof.

131. The composition of claim 97, wherein the compound is represented by one of the following Structural Formulas: 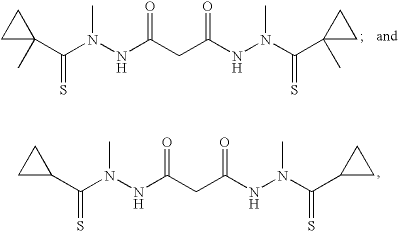
or a pharmaceutically acceptable salt thereof.

132. The composition of claim 131, wherein the compound is represented by the following Structural Formula: 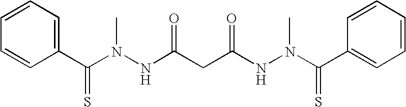
or a pharmaceutically acceptable salt thereof.

133. The composition of claim 132, wherein the compound is a disodium or a dipotassium salt.

134. The composition of claim 97, further comprising a microtubulin stabilizer selected from the group consisting of taxol; taxol analogues; Discodermolide (also known as NVP-XX-A-296); Epothilones (such as Epothilone A, Epothilone B, Epothilone C (also known as desoxyepothilone A or dEpoA); Epothilone D (also referred to as KOS-862, dEpoB, and desoxyepothilone B); Epothilone E; Epothilone F; Epothilone B N-oxide; Epothilone A N-oxide; 16-aza-epothilone B; 21-aminoepothilone B (also known as BMS-310705); 21-hydroxyepothilone D (also known as Desoxyepothilone F and dEpoF), 26-fluoroepothilone); FR-182877 (Fujisawa, also known as WS-9885B), BSF-223651 (BASF, also known as ILX-651 and LU-223651); AC-7739 (Ajinomoto, also known as AVE-8063A and CS-39.HCl); AC-7700 (Ajinomoto, also known as AVE-8062, AVE-8062A, CS-39-L-Ser.HCl, and RPR-258062A); Fijianolide B; Laulimalide; Caribaeoside; Caribaeolin; Taccalonolide; Eleutherobin; Sarcodictyin; Laulimalide; Dictyostatin-1; Jatrophane esters; and analogs and derivatives thereof, wherein the microtubulin stabilizer is substantially or completely encased in the polymeric shell

135. The composition of claim 134, wherein the microtubulin stabilizer is taxol or a taxol analog.

136-139. (canceled)

140. The composition of claim 135, wherein the taxol analog is taxotere.

141. A composition prepared by subjecting an organic phase comprising a compound represented by the following Structural Formula: 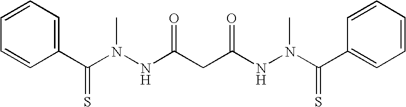
or a pharmaceutically acceptable salt thereof,
and an aqueous medium comprising a biocompatible polymer, to sonication conditions for a time sufficient to promote crosslinking of said biocompatible polymer by disulfide bonds to produce a polymeric shell encasing substantially or completely the compound; wherein the biocompatible polymer is albumin.

142-152. (canceled)

153. A composition prepared by subjecting an organic phase comprising a compound represented by the following Structural Formula: 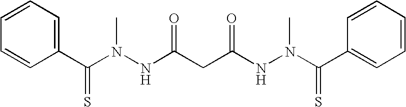
or a pharmaceutically acceptable salt thereof, and taxol or taxotere,
and an aqueous medium comprising a biocompatible polymer, to sonication conditions for a time sufficient to promote crosslinking of said biocompatible polymer by disulfide bonds to produce a polymeric shell encasing substantially or completely the compound and taxol or taxotere; wherein the biocompatible polymer is albumin.

154. (canceled)

155. A composition prepared by subjecting an organic phase comprising a compound represented by the following Structural Formula: 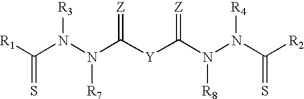
or a pharmaceutically acceptable salt or solvate thereof, wherein:
Y is a covalent bond or an optionally substituted straight chained hydrocarbyl group, or, Y, taken together with both >C=Z groups to which it is bonded, is an optionally substituted aromatic group;
R₁-R₄ are independently —H, an optionally substituted aliphatic group, an optionally substituted aryl group, or R₁ and R₃ taken together with the carbon and nitrogen atoms to which they are bonded, and/or R₂ and R₄ taken together with the carbon and nitrogen atoms to which they are bonded, form a non-aromatic heterocyclic ring optionally fused to an aromatic ring;
R₇-R₈ are independently —H, an optionally substituted aliphatic group, or an optionally substituted aryl group;
Z is O or S;
and an aqueous medium comprising a polymer, to high shear conditions in a high pressure homogenizer at a pressure in the range of about 100 up to about 100,000 psi for a time sufficient to promote crosslinking of said polymer by disulfide bonds to produce a polymeric shell encasing substantially or completely the compound.

156-189. (canceled)

190. The composition of claim 155, wherein the compound is represented by the following Structural Formula: 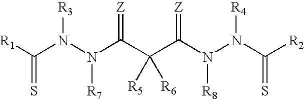
or a pharmaceutically acceptable salt or solvate thereof, wherein:
R₇-R₈ are both —H, and:
R₁ and R₂ are both phenyl, R₃ and R₄ are both methyl, and R₅ and R₆ are both —H;
R₁ and R₂ are both phenyl, R₃ and R₄ are both ethyl, and R₅ and R₆ are both —H;
R₁ and R₂ are both 4-cyanophenyl, R₃ and R₄ are both methyl, R₅ is methyl, and R₆ is —H;
R₁ and R₂ are both 4-methoxyphenyl, R₃ and R₄ are both methyl, and R₅ and R₆ are both —H;
R₁ and R₂ are both phenyl, R₃ and R₄ are both methyl, R₅ is methyl, and R₆ is —H;
R₁ and R₂ are both phenyl, R₃ and R₄ are both ethyl, R₅ is methyl, and R₆ is —H;
R₁ and R₂ are both 4-cyanophenyl, R₃ and R₄ are both methyl, and R₅ and R₆ are both —H;
R₁ and R₂ are both 2,5-dimethoxyphenyl, R₃ and R₄ are both methyl, and R₅ and R₆ are both —H;
R₁ and R₂ are both 2,5-dimethoxyphenyl, R₃ and R₄ are both methyl, R₅ is methyl, and R₆ is —H;
R₁ and R₂ are both 3-cyanophenyl, R₃ and R₄ are both methyl, and R₅ and R₆ are both —H;
R₁ and R₂ are both 3-fluorophenyl, R₃ and R₄ are both methyl, and R₅ and R₆ are both —H;
R₁ and R₂ are both 4-chlorophenyl, R₃ and R₄ are both methyl, R₅ is methyl, and R₆ is —H;
R₁ and R₂ are both 2-dimethoxyphenyl, R₃ and R₄ are both methyl, and R₅ and R₆ are both —H;
R₁ and R₂ are both 3-methoxyphenyl, R₃ and R₄ are both methyl, and R₅ and R₆ are both —H;
R₁ and R₂ are both 2,3-dimethoxyphenyl, R₃ and R₄ are both methyl, and R₅ and R₆ are both —H;
R₁ and R₂ are both 2,3-dimethoxyphenyl, R₃ and R₄ are both methyl, R₅ is methyl, and R₆ is —H;
R₁ and R₂ are both 2,5-difluorophenyl, R₃ and R₄ are both methyl, and R₅ and R₆ are both —H;
R₁ and R₂ are both 2,5-difluorophenyl, R₃ and R₄ are both methyl, R₅ is methyl, and R₆ is —H;
R₁ and R₂ are both 2,5-dichlorophenyl, R₃ and R₄ are both methyl, and R₅ and R₆ are both —H;
R₁ and R₂ are both 2,5-dimethylphenyl, R₃ and R₄ are both methyl, and R₅ and R₆ are both —H;
R₁ and R₂ are both 2,5-dimethoxyphenyl, R₃ and R₄ are both methyl, and R₅ and R₆ are both —H;
R₁ and R₂ are both phenyl, R₃ and R₄ are both methyl, and R₅ and R₆ are both —H;
R₁ and R₂ are both 2,5-dimethoxyphenyl, R₃ and R₄ are both methyl, R₅ is methyl, and R₆ is —H;
R₁ and R₂ are both cyclopropyl, R₃ and R₄ are both methyl, and R₅ and R₆ are both —H;
R₁ and R₂ are both cyclopropyl, R₃ and R₄ are both ethyl, and R₅ and R₆ are both —H;
R₁ and R₂ are both cyclopropyl, R₃ and R₄ are both methyl, R₅ is methyl, and R₆ is —H;
R₁ and R₂ are both 1-methylcyclopropyl, R₃ and R₄ are both methyl, and R₅ and R₆ are both —H;
R₁ and R₂ are both 1-methylcyclopropyl, R₃ and R₄ are both methyl, R₅ is methyl and R₆ is —H;
R₁ and R₂ are both 1-methylcyclopropyl, R₃ and R₄ are both methyl, R₅ is ethyl, and R₆ is —H;
R₁ and R₂ are both 1-methylcyclopropyl, R₃ and R₄ are both methyl, R₅ is n-propyl, and R₆ is —H;
R₁ and R₂ are both 1-methylcyclopropyl, R₃ and R₄ are both methyl, and R₅ and R₆ are both methyl;
R₁ and R₂ are both 1-methylcyclopropyl, R₃ and R₄ are both ethyl, and R₅ and R₆ are both —H;
R₁ and R₂ are both 1-methylcyclopropyl, R₃ is methyl, R₄ is ethyl, and R₅ and R₆ are both —H;
R₁ and R₂ are both 2-methylcyclopropyl, R₃ and R₄ are both methyl, and R₅ and R₆ are both —H;
R₁ and R₂ are both 2-phenylcyclopropyl, R₃ and R₄ are both methyl, and R₅ and R₆ are both —H;
R₁ and R₂ are both 1-phenylcyclopropyl, R₃ and R₄ are both methyl, and R₅ and R₆ are both —H;
R₁ and R₂ are both cyclobutyl, R₃ and R₄ are both methyl, and R₅ and R₆ are both —H;
R₁ and R₂ are both cyclopentyl, R₃ and R₄ are both methyl, and R₅ and R₆ are both —H;
R₁ and R₂ are both cyclohexyl, R₃ and R₄ are both methyl, and R₅ and R₆ are both —H;
R₁ and R₂ are both cyclohexyl, R₃ and R₄ are both phenyl, and R₅ and R₆ are both —H;
R₁ and R₂ are both methyl, R₃ and R₄ are both methyl, and R₅ and R₆ are both —H;
R₁ and R₂ are both methyl, R₃ and R₄ are both t-butyl, and R₅ and R₆ are both —H;
R₁ and R₂ are both methyl, R₃ and R₄ are both phenyl, and R₅ and R₆ are both —H;
R₁ and R₂ are both t-butyl, R₃ and R₄ are both methyl, and R₅ and R₆ are both —H;
R₁ and R₂ are ethyl, R₃ and R₄ are both methyl, and R₅ and R₆ are both —H; or
R₁ and R₂ are both n-propyl, R₃ and R₄ are both methyl, and R₅ and R₆ are both —H.

191. The composition of claim 155, wherein the compound is represented by the following Structural Formula: 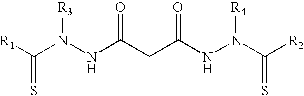
or a pharmaceutically acceptable salt thereof.

192. The composition of claim 155, wherein the compound is represented by one of the following Structural Formulas: 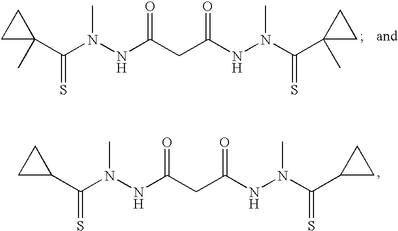
or a pharmaceutically acceptable salt thereof.

193. The composition of claim 192, wherein the compound is represented by the following Structural Formula: 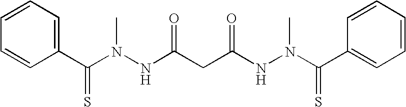
or a pharmaceutically acceptable salt thereof.

194. The composition of claim 193, wherein the compound is a disodium or a dipotassium salt.

195. The composition of claim 155, further comprising a microtubulin stabilizer selected from the group consisting of taxol; taxol analogues; Discodermolide (also known as NVP-XX-A-296); Epothilones (such as Epothilone A, Epothilone B, Epothilone C (also known as desoxyepothilone A or dEpoA); Epothilone D (also referred to as KOS-862, dEpoB, and desoxyepothilone B); Epothilone E; Epothilone F; Epothilone B N-oxide; Epothilone A N-oxide; 16-aza-epothilone B; 21-aminoepothilone B (also known as BMS-310705); 21-hydroxyepothilone D (also known as Desoxyepothilone F and dEpoF), 26-fluoroepothilone); FR-182877 (Fujisawa, also known as WS-9885B), BSF-223651 (BASF, also known as ILX-651 and LU-223651); AC-7739 (Ajinomoto, also known as AVE-8063A and CS-39.HCl); AC-7700 (Ajinomoto, also known as AVE-8062, AVE-8062A, CS-39-L-Ser.HCl, and RPR-258062A); Fijianolide B; Laulimalide; Caribaeoside; Caribaeolin; Taccalonolide; Eleutherobin; Sarcodictyin; Laulimalide; Dictyostatin-1; Jatrophane esters; and analogs and derivatives thereof, wherein the microtubulin stabilizer is substantially or completely encased in the polymeric shell

196. The composition claims 195, wherein the microtubulin stabilizer is taxol or a taxol analog.

197-200. (canceled)

201. The composition of claim 196, wherein the taxol analog is taxotere.

202. A composition prepared by subjecting an organic phase comprising a compound represented by the following Structural Formula: 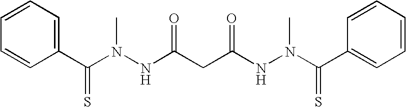
or a pharmaceutically acceptable salt thereof,
and an aqueous medium comprising a biocompatible polymer, to high shear conditions in a high pressure homogenizer at a pressure in the range of about 100 up to about 100,000 psi for a time sufficient to promote crosslinking of said polymer by disulfide bonds to produce a polymeric shell encasing substantially or completely the compound; wherein the biocompatible polymer is albumin.

203-213. (canceled)

214. A composition prepared by subjecting an organic phase comprising a compound represented by the following Structural Formula: 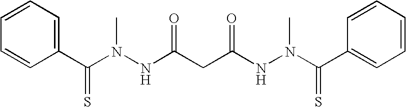
or a pharmaceutically acceptable salt thereof, and taxol or taxotere
and an aqueous medium comprising a biocompatible polymer, to high shear conditions in a high pressure homogenizer at a pressure in the range of about 100 up to about 100,000 psi for a time sufficient to promote crosslinking of said polymer by disulfide bonds to produce a polymeric shell encasing substantially or completely the compound and the taxol or taxotere; wherein the biocompatible polymer is albumin.

215. (canceled)
